(19) United States
(12) Patent Application Publication
(10) Pub. No.: US 2015/0274815 A1
(43) Pub. Date: Oct. 1, 2015

(54) TREATMENT OF CENTRAL NERVOUS SYSTEM (CNS) INJURY

(71) Applicant: THE UNITED STATES OF AMERICA, as represented by TH E SECRETARY, DEPARTMENT OF HEALTH & HUMAN SERVICE, Rockville, MD (US)

(72) Inventors: Herbert M. Geller, Chevy Chase, MD (US); Yasuhiro Katagiri, Bethesda, MD (US)

(73) Assignee: The United States of America, as represented by the Secretary, Department of Health & Human Servic, Rockville, MD (US)

(21) Appl. No.: 14/430,850

(22) PCT Filed: Sep. 25, 2013
(86) PCT No.: PCT/US13/61693

§ 371 Date: Mar. 24, 2015

Related U.S. Application Data
(60) Provisional application No. 61/705,555, filed on Sep. 25, 2012.

(51) Int. Cl. C07K 16/18; C12N 15/113; A61K 38/46

(52) U.S. Cl. CPC C07K 16/18; A61K 38/465; C12N 15/1137; C12Y 301/06012; C07K 2317/76; C12N 2310/14; C12N 2310/11; C12N 2310/531

(57) ABSTRACT

The invention features compositions for use in increasing neuron or neurite growth, including for the treatment of Central Nervous System (CNS) injury, such as human arylsulfatase B.

### RELATED APPLICATIONS

This application claims the benefit of priority under 35 U.S.C. §119(e) to U.S. Provisional Application No. 61/705,555, filed Sep. 25, 2012, which is incorporated herein by reference in its entirety.

### STATEMENT OF RIGHTS TO INVENTIONS MADE UNDER FEDERALLY SPONSORED RESEARCH

Research supporting this application was carried out by the United States of America as represented by the Secretary, Department of Health and Human Services. This research was supported by the Intramural Research Program, Developmental Neurobiology Section, Cell Biology and Physiology Center, NHLBI of the National Institute of Health. The Government has certain rights in this invention.

### BACKGROUND OF THE INVENTION

Traumatic injuries to the mammalian central nervous system (CNS) are often permanent, especially in the case of traumatic spinal cord injury. The incidence of spinal cord injury is approximately 40 cases per million in the U.S. or approximately 12,000 new cases each year. The prevalence of spinal cord injury is estimated to be approximately 265,000 persons, with a range of 232,000 to 316,000 persons. Depending on the severity of spinal cord injury and age of the individual at the time of injury, estimated costs for lifetime treatment and care of a spinal cord injury patient range from $1-5 million.

Spinal cord injury can be characterized by contusion of the neural tissue with a resultant decrease or loss of the ability of nerve tissue to properly transmit nerve impulses. Typical causes of spinal cord injury include an impact injury of some nature, a bullet wound, or certain surgical procedures involving manipulation of the spinal cord. After a spinal cord injury in the adult mammal, the inability of axons to regenerate may lead to loss of sensation, loss of motor function and/or loss of autonomic function, as well as permanent paralysis. While there is some residual growth potential of injured axons, this growth is aborted by the presence of a glial scar which forms after injury. The glial scar is both a chemical and a physical barrier to regeneration.

Chondroitin sulfate proteoglycans (CSPGs) are major components of the glial scar, and have been shown to inhibit axonal growth in many different assays. CSPGs consist of a protein core decorated by one or more glycosaminoglycan sugar side chains. These side chains consist of alternating disaccharide units of glucuronic acid and N-acetyl-galactosamine. Without being bound to a particular theory, much of the inhibitory activity of CSPGs is due to the sugar side chains, as axonal regrowth is facilitated when CSPGs are digested with the enzyme chondroitinase ABC (cABC).

At present, no effective treatment exists for central nervous system injury. For example, cABC is being studied as a treatment for spinal cord injury, but has not entered clinical trials. Although cABC has the potential to be useful for treating spinal cord injury, cABC is a bacterial enzyme and may cause immune system issues if used repeatedly Inhibition of Rho GTPase has been proposed as a potential treatment for CNS axon regrowth. However, an inhibitor of Rho GTPase failed to demonstrate efficacy in a clinical trial. Other strategies involving activated macrophages, stem cells, receptor blocking agents (e.g., antagonists, Riluzole, antibodies to NOGO receptor) and cAMP-PDE inhibitors have been proposed, but have not yielded therapies. Accordingly, new methods of treatment for central nervous system injuries are urgently required.

### SUMMARY OF THE INVENTION

The present invention features compositions and methods for the treatment of central nervous system injury or trauma (e.g., spinal cord injury) involving the use of 4-sulfatase (e.g., arylsulfatase B (ARSB), 4-sulfatase).

In one aspect, the invention provides a method of treating or preventing a central nervous system injury in a subject, the method involving administering to the subject a therapeutically effective amount of an agent that reduces the amount or activity of a 4-sulfated GalNAc in a chondroitin glycosaminoglycan chain, thereby treating the central nervous system injury.

In another aspect, the invention provides a method of treating or preventing a glial scar in a subject, the method involving administering to the subject a therapeutically effective amount of an agent that reduces the amount or activity of a 4-sulfated GalNAc in a chondroitin glycosaminoglycan chain, thereby treating the glial scar. For example, the glial scar is reduced by at least 1%, at least 2%, at least 3%, at least 4%, at least 5%, at least 10%, at least 15%, at least 20%, at least 25%, at least 30%, at least 40%, at least 50%, at least 60%, at least 70%, at least 80%, at least 90%, or at least 99% after treatment with the compounds of the invention compared to the glial scar prior to treatment.

In still another aspect, the invention provides a method of increasing neuron or neurite growth, proliferation, or migration, the method involving contacting a neuron with an effective amount of an agent that reduces the amount or activity of a 4-sulfated GalNAc in a chondroitin glycosaminoglycan chain, thereby increasing neuron or neurite growth, proliferation, or migration. In various embodiments, the neuron or neurite growth, proliferation, or migration is in a subject.

In various embodiments of any of the aspects delineated herein, the subject has had or is at risk of having a central nervous system injury, stroke, or myocardial infarction. In various embodiments of any of the aspects delineated herein, the subject is a mammal (e.g., human).

The methods described herein increase neuron or neurite growth, proliferation, or migration. Specifically, the methods described herein increase the number of axons or axonal length in or through a spinal cord lesion of the subject. For example, the methods described herein increase the number of axons at or in proximity to the spinal cord lesion by at least 1%, at least 5%, at least 10%, at least 20%, at least 30%, at least 40%, at least 50%, at least 60%, at least 70%, at least 80%, at least 90%, or at least 100% compared to the number of axons at or in proximity to the spinal cord lesion in the absence of compound, e.g., ARSB, administration. Similarly, the methods described herein increase axonal length in or through the spinal cord lesion by at least 1%, at least 5%, at least 10%, at least 20%, at least 30%, at least 40%, at least 50%, at least 60%, at least 70%, at least 80%, at least 90%, or at least 100% compared to the axonal length in or through the spinal cord lesion in the absence of compound, e.g., ARSB, administration.

In some cases, the methods described herein treat spinal cord injury by improving the function of various tissues and/or organs. For example, the methods described herein result in an improvement in cognitive skills, breathing function, diaphragm function, arm function, shoulder function, wrist function, hand function, finger function, abdominal muscle function, leg function, hip function, feet function, toe function, bladder function, anal function, and/or sexual function. In another aspect, the methods described herein alleviate pain and/or paralysis. For example, the methods described herein result in 1%-100% recovery of motor/sensory function after spinal cord injury, e.g., about 2%, about 3%, about 4%, about 5%, about 6%, about 7%, about 8%, about 9%, about 10%, about 15%, about 20%, about 25%, about 30%, about 35%, about 40%, about 45%, about 50%, about 60%, about 70%, about 80%, about 90%, about 95% or about 99% recovery of motor/sensory function after spinal cord injury.

In various embodiments of any of the aspects delineated herein, the central nervous system injury is a spinal cord injury, contusion injury to the central nervous system, or non-contusion injury to the central nervous system.

The methods involve administering to a subject having a neuron injury an effective amount of a therapeutic agent of the invention, e.g., a composition comprising human arylsulfatase B. Preferably, such agents are administered as part of a composition additionally comprising a pharmaceutically acceptable carrier. Therapeutic agents may be administered locally at the site of injury (e.g., nucleic acids via liposomal or viral delivery, polypeptides, antibodies) or systemically (e.g., small molecule inhibitors, antibodies) as to be effective, as is known to those skilled in the art. For example, ARSB (polypeptide or nucleotide) is injected directly into and around a spinal cord lesion to promote axon regeneration within the spinal cord lesion. Alternatively, ARSB is administered intravenously.

In some cases, ARSB is administered in a vector, e.g., a lentivial vector or an adenoviral vector, which has been engineered to express ARSB. Alternatively, ARSB is administered without a vector. For example, recombinant human ARSB (e.g., NAGLAZYME®) is administered at about 0.1 mg/kg to 10 mg/kg of body weight, e.g., about 0.5 mg/kg, 1 mg/kg, 1.5 mg/kg, 2 mg/kg, 2.5 mg/kg, 3 mg/kg, 3.5 mg/kg, 4 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, or 10 mg/kg of body weight. Human ARSB is administered once per month, twice per month, twice per week, once per week, twice per day, or once per day. Of course, the therapeutically effective amount may be optimized via upward or downward adjustments, as is routinely done in such treatment protocols, depending upon the specific condition and severity thereof.

In various embodiments of any of the aspects delineated herein, the 4-sulfated GalNAc reduces neuron or neurite growth, proliferation, or migration. In particular embodiments, the 4-sulfated GalNAc is at a non-reducing end of a chondroitin glycosaminoglycan chain.

In various embodiments of any of the aspects delineated herein, the agent reduces 4-sulfation of a chondroitin glycosaminoglycan chain. In various embodiments of any of the aspects delineated herein, the agent has 4-sulfatase activity (e.g., a human or bacterial GalNAc 4-sulfatase). In particular embodiments, the GalNAc 4-sulfatase activity is arylsufatase B. In various embodiments of any of the aspects delineated herein, the agent inhibits a chondroitin sulfate transferase. In various embodiments, the chondroitin sulfate transferase is selected from the group consisting of chondroitin 4-O-sulfotransferase 1 (CHST11), chondroitin 4-O-sulfotransferase 2 (CHST12), chondroitin 4-O-sulfotransferase 3 (CHST13), dermatan 4-O-sulfotransferase 1 (CHST14), chondroitin 6-O-sulfotransferase 1 (CHST3), uronyl 2-sulfotransferase (UST), carbohydrate (N-acetylgalactosamine 4-sulfate 6-O) sulfotransferase 15 (CHST15). In certain embodiments, the agent specifically binds a 4-sulfated GalNAc.

In various embodiments of any of the aspects delineated herein, the agent increases neuron or neurite growth, proliferation, or migration. In particular embodiments, the neurite is a dendrite or axon. In certain embodiments, the agent is administered locally (e.g., intrathecal or topical administration). In various embodiments of any of the aspects delineated herein, the agent is an inhibitory nucleic acid molecule (e.g., an antisense molecule, an siRNA, or an shRNA). In various embodiments of any of the aspects delineated herein, the agent is an antibody or fragment thereof. In particular embodiments, the antibody is monoclonal or polyclonal.

Other features and advantages of the invention will be apparent from the detailed description, and from the claims.

### DEFINITIONS

By “4-sulfatase” is meant an enzyme having hydrolytic activity to catalyze the removal of a sulfate moiety from the C4 position of N-acetylgalactosamine. An exemplary 4-sulfatase of the invention is Arylsulfatase B (ARSB), preferably human Arylsulfatase B.

By “axon” is meant a long, slender projection of a nerve cell, or neuron, that typically conducts electrical impulses away from the neuron's cell body. Axon dysfunction causes many inherited and acquired neurological disorders which can affect both the peripheral and central neurons.

By “binding to” a molecule is meant having a physicochemical affinity for that molecule.

By “ameliorate” is meant decrease, suppress, attenuate, diminish, arrest, or stabilize the development or progression of a disease.

By “compound” is meant any small molecule chemical compound, antibody, nucleic acid molecule, or polypeptide, or fragments thereof.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

By “dendrite” is meant a branched projection of a neuron that acts to conduct the electrochemical stimulation received from other neural cells to the cell body, or soma, of the neuron from which the dendrites project.

By “disease” is meant any condition or disorder that damages or interferes with the normal function of a cell, tissue, or organ.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “inhibitory nucleic acid molecule” is meant a polynucleotide that disrupts the expression of a target nucleic acid molecule or an encoded polypeptide. Exemplary inhibitory nucleic acid molecules include, but are not limited to, shRNAs, siRNAs, antisense nucleic acid molecules, and analogs thereof.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder.

By “neuron” is meant an electrically excitable cell that processes and transmits information by electrical and chemical signaling. Chemical signaling occurs via synapses, specialized connections with other cells. Neurons connect to each other to form neural networks. Neurons are the core components of the nervous system, which includes the brain, spinal cord, and peripheral ganglia.

By “neurite” is meant a projection from the cell body of a neuron (e.g., an axon or a dendrite). The term is frequently used when speaking of developing neurons, especially of cells in culture, because it can be difficult to tell axons from dendrites before differentiation is complete.

By “protein” or “polypeptide” or “peptide” is meant any chain of more than two natural or unnatural amino acids, regardless of post-translational modification (e.g., glycosylation or phosphorylation), constituting all or part of a naturally-occurring or non-naturally occurring polypeptide or peptide, as is described herein.

By “subject” is meant a mammal, including, but not limited to, a human or non-human mammal, such as a bovine, equine, canine, ovine, or feline.

The term “reduce” or “increase” is meant to alter negatively or positively, respectively, by at least 5%. An alteration may be by 5%, 10%, 25%, 30%, 50%, 75%, or even by 100%.

By “reference” is meant a standard or control condition. In one embodiment, the effect of an agent on a cell is compared to the effect of the agent on a control cell.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “selectively” is meant the ability to affect the activity or expression of a target molecule without affecting the activity or expression of a non-target molecule. For example, arylsulfatase B selectively reduces the levels of 4-sulfate modifications at non-reducing ends of chondroitin sulfate glycosaminoglycan chains compared to bacterial 4-sulfatase.

By “specifically binds” is meant a compound or antibody that recognizes and binds a polypeptide of the invention, but which does not substantially recognize and bind other molecules in a sample.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and most preferably 90%, 95%, 96%, 97%, 98%, or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

“Therapeutic agent” means a substance that has the potential of affecting the function of an organism. Such a compound may be, for example, a naturally occurring, semi-synthetic, or synthetic agent. For example, an agent may be a drug that targets a specific function of an organism or an antibiotic. A therapeutic agent may decrease, suppress, attenuate, diminish, arrest, or stabilize the development or progression of disease, disorder, or infection in a eukaryotic host organism.

By the terms “effective amount” and “therapeutically effective amount” of a formulation or formulation component is meant a sufficient amount of the formulation or component, alone or in a combination, to provide the desired effect. For example, by “an effective amount” is meant an amount of a compound, alone or in a combination, required to reduce or prevent CNS injury, e.g., spinal cord injury, in a mammal. Of course, the therapeutically effective amount may be optimized via upward or downward adjustments, as is routinely done in such treatment protocols, depending upon the specific condition and severity thereof.

### BRIEF DESCRIPTION OF THE DRAWINGS

FIG. 1 is a schematic depicting models of nervous system response to injury. In contrast to the peripheral nervous system (PNS), the mammalian central nervous system (CNS) does not regenerate following injury. Without being bound to a particular theory, formation of a glial scar at the site of trauma prevents neuronal growth and/or creates an environment that inhibits neuronal growth.

FIGS. 2A and 2B depict glial scar staining for CSPGs and the astrocyte marker glial fibrillary acidic protein. Chondroitin sulfate proteoglycans (CSPG) are present at the site of CNS injury. Chondroitin sulfate proteoglycans consist of a protein core and GAG side chains of alternating Glucuronic acid (GlcA) and N-Acetylgalactosamine (GalNAc) monomers, which may be modified by a sulfate moiety.

FIG. 3 is a schematic depicting interactions of putative receptors, binding sites, and signal transduction pathways involved in neuronal growth pathways, including Nogo receptors (NgR) and Rho GTPase.

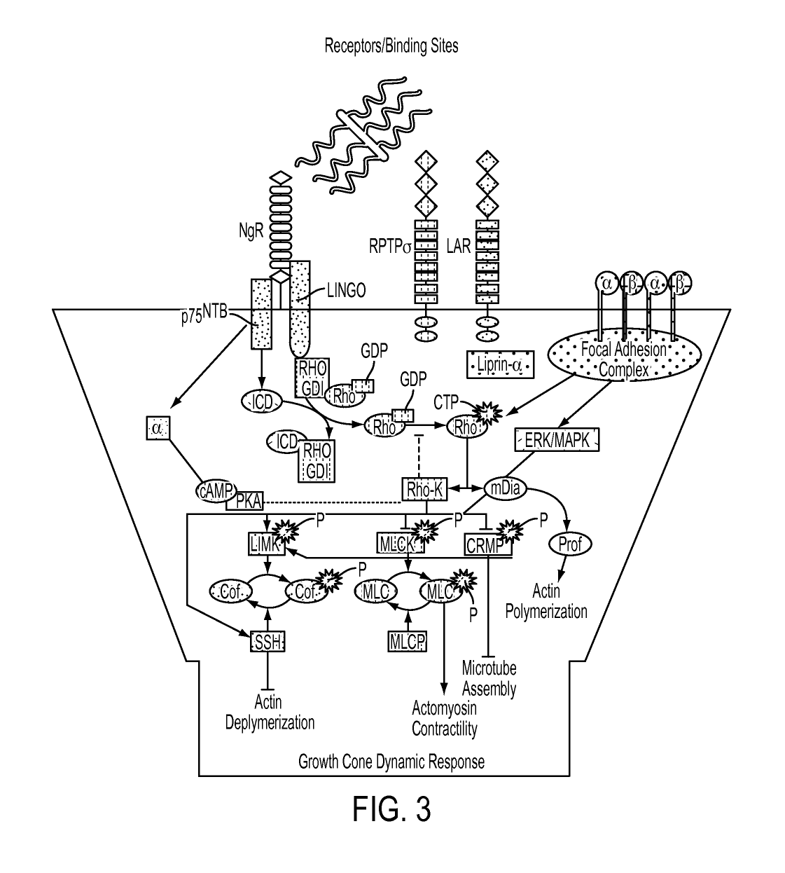

FIG. 4 is a schematic depicting the biosynthesis of chondroitin sulfate. Chondroitin sulfate chains are linked by xylose to serine residues in core proteins. Xylosyltransferase (XT) initiates the process using UDP-xylose as a donor. To the Xyl residue are sequentially transferred two Gal and one GlcA residues (catalyzed by β1-4 galactosyl-, β1-3 galactosyl-, and β1-3 glucuronosyltransferase enzymes, respectively, form a linkage tetrasaccharide). Addition of β1-4GalNAc results in initiation of chondroitin sulfate. Chondroitin polymerizing factor (ChPF) and chondroitin synthases (CSS) add GlcA-GalNAc disaccharides to the glycosaminoglycan chain. The glycosaminoglycan chain is modified by chondroitin sulfate transferases (e.g., chondroitin 4-O-sulfotransferase 1/CHST11, chondroitin 4-O-sulfotransferase 2/CHST12, chondroitin 4-O-sulfotransferase 3/CHST13, dermatan 4-O-sulfotransferase 1/CHST14, chondroitin 6-O-sulfotransferase 1/CHST3, uronyl 2-sulfotransferase/UST, carbohydrate (N-acetylgalactosamine 4-sulfate 6-O) sulfotransferase 15/CHST15), which contribute to glycosaminoglycan chain heterogeneity.

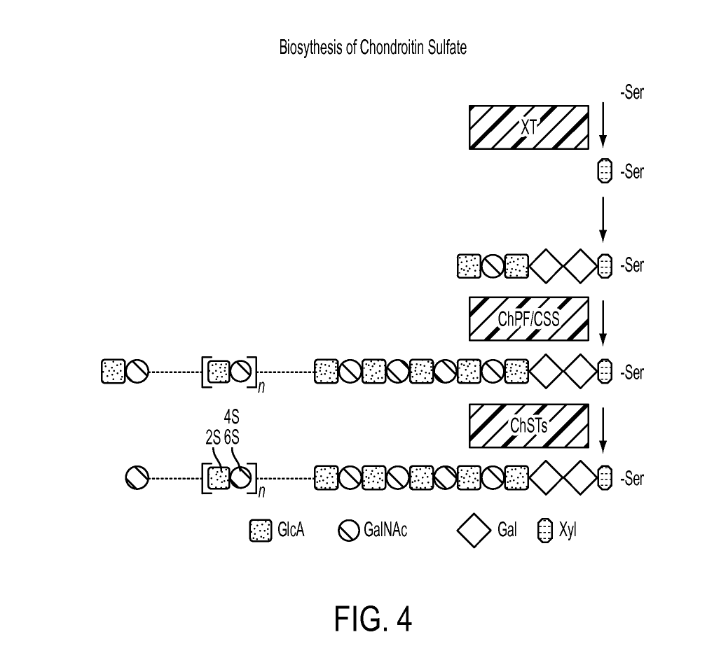

FIG. 5 depicts various sulfate modifications of chondroitin sulfate that contribute to heterogeneity of glycosaminoglycan chain dissacharides. Disaccharides with no sulfation or with three sulfates (GlcA(2-O-sulfate)-GalNAc(4,6-O-sulfate)) are known to exist but are not shown in the figure.

FIG. 6 is a schematic depicting blockade of chondroitin sulfate biosynthesis. Blocking steps of chondroitin sulfate biosynthesis to inhibit biosynthesis of chondroitin sulfate has been proposed to treat or prevent glial scar formation and/or increase neuronal growth at sites of injury Inhibiting chondroitin polymerizing factor (ChPF) and chondroitin synthases (CSS) (e.g., using an siRNA) blocks glycosaminglycan chain formation. Sulfation of glycosaminoglycans, including 4-sulfation, may be inhibited by chlorate.

FIG. 7 is a table that lists various published studies showing conflicting results regarding the effect of chondroitin sulfate modifications on neuronal growth.

FIG. 8 is a schematic depicting the activity of chondroitinase in degrading chrondroitin sulfate sugars. Degradation of chondroitin sulfate (e.g., by chondroitinase) has been proposed to reduce the effects of glial scar and/or increase neuronal growth at sites of injury.

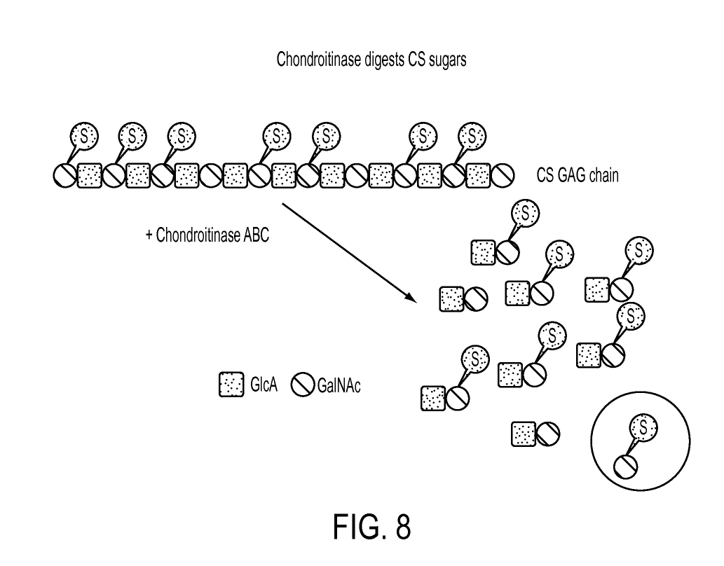

FIG. 9 shows that partial digestion is effective for decreasing repellant activity of neurons by chondroitin sulfate.

FIG. 10 depicts immunofluorescence images of acute spinal cord injury showing increased staining of 4-sulfation of chondroitin sulfate at the site of injury.

FIG. 11 depicts immunofluorescence images showing that 4-S chondroitin sulfate chains formed a boundary for neurons and/or has an activity that repelled neurons.

FIG. 12 depicts immunofluorescence images showing that treatment of chondroitin sulfate chains with 4-sulfatase decreased the neuron repellant and/or boundary activity of chondroitin sulfate.

FIG. 13 is a schematic depicting specific blockade of 4-S chondroitin sulfate biosynthesis. Blocking 4-S modification of chondroitin sulfate has the potential to treat or prevent glial scar formation and/or increase neuronal growth at sites of injury. Inhibiting chondroitin sulfate transferases (CHST11, 12, 13, 14, 15)(e.g., using an siRNA) blocks 4-sulfation of glycosaminglycan chains.

FIG. 14 is a schematic depicting the change in overall sulfation is small when chondroitin sulfatase is treated with 4-sulfatase. Treatment of chondroitin sulfate with 4-sulfatase decreased overall sulfation by about 6% compared to a control without treatment.

FIG. 15 is a schematic depicting that sulfation of chondroitin may provide signals affecting neuronal growth. Without being bound to a particular theory, the 4-S modification of chondroitin sulfate may provide a signal or activity that prevents or decreases neuronal growth.

FIG. 16 is a chart depicting that treatment with arylsulfatase B (ARSB) reduced chondroitin sulfate neuron inhibitory activity compared to control without treatment.

FIG. 17 depicts results of a study showing that knockdown of arylsulfatase B (ARSB) expression decreased neuronal growth and/or increased astrocyte inhibition compared to control as measured by neurite length.

FIG. 18 depicts results of a study showing that overexpression of arylsulfatase B (ARSB) expression increased neuronal growth and/or decreased astrocyte inhibition compared to control as measured by neurite length.

FIG. 19 depicts results of a study showing that lentiviral arylsulfatase B (ARSB) expression increased axon length at and around the lesion border compared to control as measured by fiber index.

### DETAILED DESCRIPTION OF THE INVENTION

The invention features compositions and methods that are useful for treating trauma or injuries to the mammalian Central Nervous System (CNS) (e.g., traumatic spinal cord injury or traumatic brain injury). In particular, the invention provides compositions effective in reducing the biosynthesis of chondroitin 4-sulfate (e.g., an inhibitory nucleic acid targeting a gene of the chondroitin sulfate biosynthetic pathway) or increasing the degradation of chondroitin 4-sulfate (e.g., 4-sulfatase); and methods of using the composition for the treatment of injury to the mammalian Central Nervous System (CNS) and/or neuronal growth at a site of injury. In particular embodiments, the invention provides compositions comprising a human 4-sulfatase (e.g., arylsulfatase B).

The invention is based, at least in part, on several surprising discoveries: that 4-sulfate modified chondroitin sulfate chains formed a boundary and/or decreased neuronal growth; and treatment of chondroitin sulfate chains decreased the inhibitory or repellant activity on neuronal growth. These findings indicate that 4-sulfate modified chondroitin sulfate chains inhibit or repel neuronal growth (e.g., by providing an inhibitory signal). In particular, treatment with arylsulfatase B (ARSB), which targets 4-sulfate modifications at the non-reducing end of chondroitin sulfate glycosaminoglycan chains (the end that is furthest from the protein core), was shown to be effective in reducing repellent activity of chondroitin sulfate, although reducing total 4-sulfation of chondroitin sulfate was also effective. It is appreciated that these discoveries are applicable to treatment of nervous system trauma that result in glial scarring. As 4-sulfate modified chondroitin sulfate chains are present in the glial scar, agents that decrease 4-sulfation in chondroitin sulfate chains are expected to be effective for the treatment of injury to the mammalian Central Nervous System (CNS) and/or for increasing neuronal growth at a site of injury. The compositions and methods of the invention may be used in the treatment of other types of CNS injury which result in a glial scar, such as stroke. Recent evidence indicates that CSPGs prevent reinnervation of injured cardiac tissue after myocardial infarction (MI). The compositions and methods of the invention are expected to be useful for neuronal growth at a site of injury or trauma.

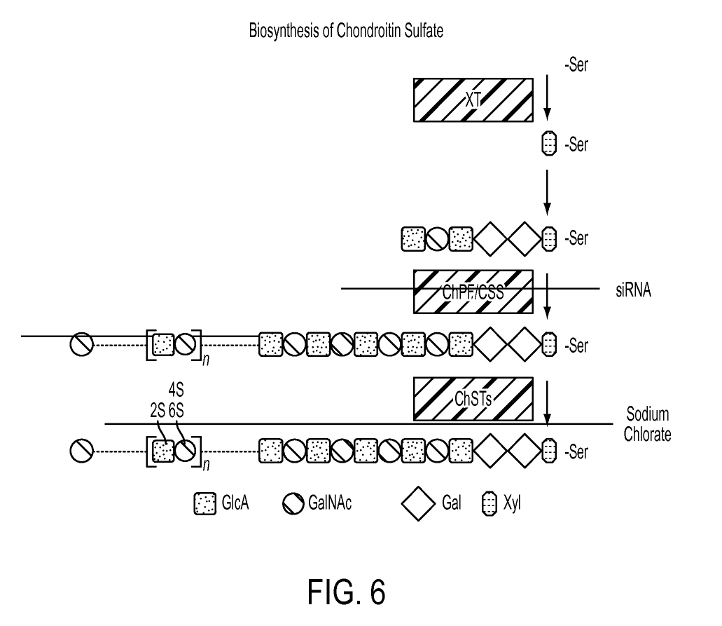

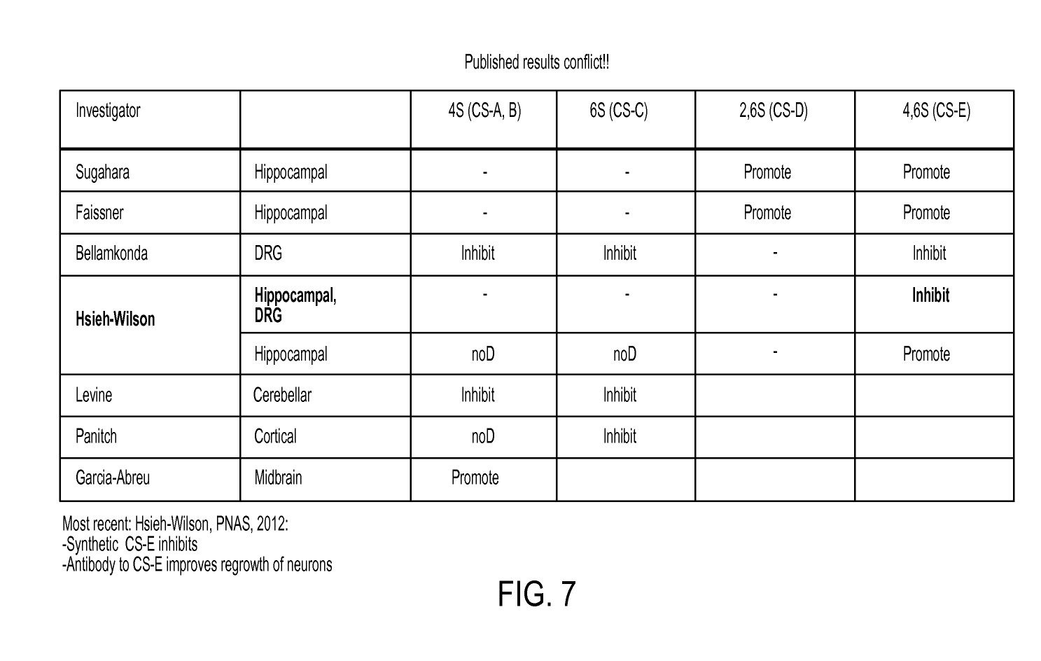

Prior to the invention described herein, there was no effective treatment for central nervous system injury Inhibition of the biosynthesis of chondroitin sulfate was proposed to treat or prevent glial scar formation and/or increase neuronal growth at sites of injury (FIG. 6); however, published studies conflicted regarding the effect of chondroitin sulfate modifications on neuronal growth (FIG. 7). In one example, EP Publication No. 1480674 (WO 03/074080) described the utilization of chondroitinase ABC to block the synthesis of chondroitin sulphate proteoglycans to promote neuronal plasticity in the CNS; however, cABC has not entered clinical trials, and it is unclear whether cABC would be effective in humans, as cABC is a bacterial enzyme that might cause human immune system issues.

Prior to the invention described herein, inhibition of Rho GTPase was also proposed as a potential treatment for CNS axon regrowth; however, an inhibitor of Rho GTPase failed to demonstrate therapeutic efficacy in a clinical trial. Similarly, other strategies involving activated macrophages, stem cells, receptor blocking agents (e.g., antagonists, Riluzole, antibodies to NOGO receptor), and cAMP-PDE inhibitors also failed to yield therapeutic results.

As described herein, 4-sulfate-modified chondroitin sulfate chains (i.e., 4-sulfated GalNAc) in glial scars inhibit neurite outgrowth and repel axons. Although Wang et al., 2008 J Cell Sci, 121(Pt 18):3083-3091 described that chondroitin-4-sulfation negatively regulates axonal guidance and growth, prior to the invention described herein, it was unknown whether specific inhibition of 4-sulfation in chondroitin sulfate chains present in the glial scar would be sufficient to treat CNS injury and/or increase neuronal growth at a site of injury.

Described herein is the utilization of a chondroitin-4-sulfate modifier, arylsulfatase B (ARSB), e.g., human ARSB, to reduce the amount or activity of a 4-sulfated GalNAc in a chondroitin glycosamino glycan chain to treat CNS injury. Arylsulfatase B is an N-acetylgalactosamine (GalNAc) 4-sulfatase (Enzyme Nomenclature Number 3.1.6.12), which localizes to the lysosome where it specifically catalyzes the removal of the C4 sulfate group from GalNAc at the nonreducing terminus of chondroitin sulfate/dermatan sulfate gylcosylaminoglycan (GAG) chains during lysosomal degradation. While bacterial 4-sulfatases may remove the 4-sulfate group from GlcA β1-3GalNAc (4S), ARSB is unable to do so due to the presence of GlcA at the non-reducing end of chondroitin sulfate glycosaminoglycan chains. Thus, in contrast to chondroitinase ABC, which generally degrades many substrates within chondroitin sulfate glycosaminoglycan chain disaccharides, ARSB specifically targets 4-sulfated GalNAc at the non-reducing ends of CS-GAGs. ARSB also has a more robust chemical stability and a reduced immunogenicity compared to chondroitinase ABC.

The human ARSB described herein also acts on long chain glycosaminoglycan substrates. By contrast, bacterial sulfatases do not remove the 4-sulfate group from long GAG chains effectively. Rather, bacterial sulfatases act only on short chain glycosaminoglycan chain substrates (e.g., disaccharides).

### Spinal Cord Injury (SCI)

The spinal cord is made up of nerve fibers. Damage to the central nervous system, including the spinal cord results in a loss of function. Depending upon the type of injury to the central nervous system, the loss of function may manifest itself in loss of sensory, motor or autonomic function or a combination thereof. Sensory functions include the ability to feel sensations, like pain. Motor functions include the ability to voluntarily move your body. Autonomic functions include involuntary body functions, for example the ability to sweat and breathe.

Common types of spinal cord injuries (SCI) include contusions (bruising of the spinal cord) and compression injuries (caused by prolonged pressure on the spinal cord). In contusion and compression injuries, a cavity or hole often forms in the center of the spinal cord. Unlike neurons of the peripheral nervous system (PNS), neurons of the central nervous system (CNS) do not regenerate after injury.

Spinal cord injury can be characterized by contusion of the neural tissue with a resultant decrease or loss of the ability of nerve tissue to properly transmit nerve impulses. The usual cause is due to an impact injury of some nature, but it may also occur due to gunshot wounds or during the manipulation of the spinal cord in certain surgical procedures. After a spinal cord injury in the adult mammal, the inability of axons to regenerate may lead to loss of sensation, loss of motor function and/or loss of autonomic function, as well as permanent paralysis.

One reason that neurons fail to regenerate is their inability to traverse the glial scar that develops following a spinal cord injury. The injury-induced lesion will develop glial scarring, which contains extracellular matrix molecules including chondroitin sulfate proteoglycans (CSPGs). CSPGs inhibit nerve tissue growth in vitro and nerve tissue regeneration at CSPGs rich regions in vivo.

The symptoms associated with spinal cord injury vary widely and depend upon the location of the spinal cord damage. Sensory and motor impairments following SCI are often described at various levels of “incomplete,” which vary from having little or no effect on the patient to a “complete” injury, which indicates a total loss of function. See, e.g., International Standards for the Neurological Classification of Spinal Cord Injury (ISNCSCI), J Spinal Cord Med. 2011 November; 34(6):547-54, incorporated herein by reference. The classification is based on neurological responses, touch and pinprick sensations tested in each dermatome, and strength of the muscles that control ten key motions on both sides of the body, including hip flexion (L2), shoulder shrug (C4), elbow flexion (CS), wrist extension (C6), and elbow extension (C7). Traumatic spinal cord injury is classified into five categories on the American Spinal Injury Association (ASIA) Impairment Scale: “A” indicates a “complete” spinal cord injury where no motor or sensory function is preserved in the sacral segments S4-S5. “B” indicates an “incomplete” spinal cord injury where sensory, but not motor function is preserved below the neurological level and includes the sacral segments S4-S5. This is typically a transient phase and if the person recovers any motor function below the neurological level, that person essentially becomes a motor incomplete, i.e. ASIA C or D. “C” indicates an “incomplete” spinal cord injury, wherein motor function is preserved below the neurological level and more than half of key muscles below the neurological level have a muscle grade of less than 3, which indicates active movement with full range of motion against gravity. “D” indicates an “incomplete” spinal cord injury where motor function is preserved below the neurological level and at least half of the key muscles below the neurological level have a muscle grade of 3 or more. Finally, “E” indicates “normal,” wherein motor and sensory scores are normal.

Determining the exact “level” of injury is critical in determining accurate predictions about the specific parts of the body that may be affected by paralysis and loss of function. The “level” is assigned according to the location of the injury by the vertebra of the spinal column Generally, the lower the level of vertebrae injury, the less severe the effects of paralysis, etc. While the prognosis of complete injuries is generally predictable since recovery is rare, the symptoms of incomplete injuries can vary, and it is often difficult to make an accurate prediction of the outcome. Cervical (neck) injuries usually result in full or partial tetraplegia (quadriplegia). However, depending on the specific location and severity of trauma, limited function may be retained. For example, injuries at the C-1/C-2vertebrae levels will often result in loss of breathing, necessitating mechanical ventilators or phrenic nerve pacing. Injury to the C3 vertebrae and above typically results in loss of diaphragm function, necessitating the use of a ventilator for breathing, while injury to C4 results in significant loss of function at the biceps and shoulders. Injury to C5 results in potential loss of function at the biceps and shoulders and complete loss of function at the wrists and hands, while injury to C6 results in limited wrist control and complete loss of hand function. Injury to C7 and T1 results in lack of dexterity in the hands and fingers, but allows for limited use of arms. Patients with complete injuries above C7 typically cannot handle activities of daily living. As such, functioning independently is difficult or impossible. Additional signs and symptoms of cervical injuries include: inability or reduced ability to regulate heart rate, blood pressure, sweating and hence body temperature; and autonomic dysreflexia or abnormal increases in blood pressure, sweating, and other autonomic responses to pain or sensory disturbances.

Complete injuries at or below the thoracic spinal levels result in paraplegia. Functions of the hands, arms, neck, and breathing are usually not affected. For example, injury to T1 to T8 results in the inability to control the abdominal muscles. Accordingly, trunk stability is affected. Injury to T9 to T12 results in partial loss of trunk and abdominal muscle control. Typically lesions above the T6 spinal cord level can result in Autonomic Dysreflexia.

The effects of injuries to the lumbar or sacral regions of the spinal cord are decreased control of the legs and hips, urinary system, and anus. Bowel and bladder function is regulated by the sacral region of the spine. In that regard, it is very common to experience dysfunction of the bowel and bladder, including infections of the bladder and anal incontinence, after traumatic injury. Sexual function is also associated with the sacral spinal segments, and is often affected after injury.

Treatment of spinal cord injuries starts with restraining the spine and controlling inflammation to prevent further damage. The actual treatment can vary widely depending on the location and extent of the injury. In many cases, spinal cord injuries require substantial physical therapy and rehabilitation, especially if the patient's injury interferes with activities of daily life.

The methods described herein treat spinal cord injury by improving the function of various tissues and/or organs. For example, the methods described herein improve breathing function, diaphragm function, arm function, shoulder function, wrist function, hand function, finger function, abdominal muscle function, leg function, hip function, feet function, toe function, bladder function, anal function, and/or sexual function.

### Nerve Growth and the Extracellular Matrix (ECM)

A number of molecules, and specified regions thereof, have been implicated in the ability to support the sprouting of neurites from a neuronal cell, a process also referred to as neurite outgrowth. The term neurite refers to both axon and dendrite structures. The process of forming neurites is essential in neural development and regeneration, especially after physical injury or disease has damaged neuronal cells. Neurites elongate profusely during development both in the central and peripheral nervous systems of all animal species. This phenomenon pertains to both axons and dendrites.

Various polypeptides, especially cell adhesion molecules (CAMs), have been known to promote neural cell growth. While early efforts in this area of research concentrated on the adhesion-promoting extracellular matrix protein fibronectin (FN), other polypeptides have also been found to promote neural growth. For example, U.S. Pat. No. 5,792,743 discloses novel polypeptides and methods for promoting neural growth in the CNS of a mammal by administering a soluble neural CAM, a fragment thereof, or a Fc-fusion product thereof. U.S. Pat. No. 6,313,265 discloses synthetic polypeptides containing the pharmacologically active regions of CAMs that can be used in promoting nerve regeneration and repair in both peripheral nerve injuries as well as lesions in the CNS. While helpful, the use of regenerative proteins alone may not be sufficient to effect repair of a damaged nervous system.

Proteoglycans, major constituents of the extracellular matrix, are known to be present in large amounts in glial scar tissue and to inhibit recovery following spinal cord injuries (Fawcett & Asher, 1999). Enzymes that are capable of digesting glial scar tissue are being studied for their use as spinal cord injury (SCI) therapeutics. Chondroitinase ABC (EC 4.2.2.4; cABC) is a bacterial enzyme that catalyzes the digestion of sulfated chondroitin and dermatan side chains of proteoglycans. This enzyme has been shown to promote functional recovery after spinal cord injury (Bradbury et al., 2002; Caggiano et al., 2005).

The action of enzymes and other polypeptides which degrade components of the extracellular matrix and basement membranes may facilitate the events of neural repair by a variety of mechanisms, including the release of bound cytokines and by increasing the permeability of the matrix, thereby enhancing the mobility of mediator molecules, growth factors and chemotactic agents, as well as the cells involved in the healing process. For example, U.S. Pat. No. 5,997,863 discloses the use of glycosaminoglycans to manipulate cell proliferation and promote wound healing.

Components of the inhibitory CSPGs have been identified as the glycosaminoglycans, chondroitin sulfate (CS) and dermatan sulfate (DS). Removal of these inhibitory molecules would allow neurites to regenerate and reinnervate an area after physical injury or disease, as well as to allow for the recovery of sensory, motor and autonomic functions.

Chondroitinases can lyse and degrade CSPGs including, CS and DS. In one study, chondroitinase ABC removed glycosaminoglycan (GAG) chains in and around lesioned areas of rat CNS in vivo. The degradation of GAGs promoted expression of a growth-associated protein, GAP-43, indicating an increase in the ability of axons in the treated area to regenerate.

### Chondroitin Sulfate (CS)

Chondroitin sulfates (CS) are sulfated polysaccharides in linear chains of a repeated dissacharides. They range in molecular weight from about 10,000 to over 100,000 Da. Chondroitin sulfate exists in different isomers designated by the appended letters A, B, and C (Hoffman et al., 1958). The repeating units are composed of uronic acid (GlcA or IdoA) and N-acetyl-galactosamine, and these polysaccharide chains are called glycosaminoglycans, typically abbreviated as GAG. GAG chains are somewhat heterogeneous. The letters refer to the disaccharide units within the chain. Although GAG chain species have different repeating disaccharide regions, they are covalently bound through the so-called linkage region tetrasaccharide sequence to the serine residue in the GAG attachment consensus sequence (Glu/Asp-X-Ser-Gly) of respective core proteins. Chondroitin sulfate A and C (CS-A, CS-C) are the most abundant GAGs and are found in cartilage, bone and heart valves. Chondroitin sulfate B (CS-B, or, alternatively, dermatan sulfate) is expressed mostly in skin, blood vessels, and heart valves. CS chains in the mammalian nervous system contain CS-A, CS-B CS-C, CS-D and CS-E units. The results described herein indicate removal of 4-sulfation at the non-reducing terminal is important for neurite growth. Without being bound to a particular theory, the stoichiometry of 4-sulfation has a potential role in regulating its inhibitory activity of CS GAGs. Increasing 4-sulfatase activity by 50%, 60%, 70%, 80%, 90%, 95%, 96%, 97%, 98%, 99% or more, up to 100%, favors the removal of 4-sulfate. Decreasing overall sulfation by at least about 5%, 10%, 20%, 30%, 40%, 50%, 60%, 70%, 80%, 90%, or more, up to 100% was shown to be useful. Other parts of the GAG chain are potentially important for other functions, as reducing total 4-sulfation of chondroitin sulfate was also effective in reducing repellent activity of chondroitin sulfate.

Chondroitin sulfate chains are linked by xylose to serine residues in core proteins. Xylosyltransferase (XT) initiates the process using UDP-xylose as a donor. Sites of xylosylation have a glycine residue carboxy-terminal to the serine attachment site. At least two acidic amino acid residues are located on one or both sides of the serine residue, usually within a few amino acids. Xylosylation is an incomplete process in some proteoglycans, which may explain why proteoglycans with multiple potential attachment sites contain different numbers of chains in different cells. Variation in the degree of glycosaminoglycan substitution also might result from low levels of UDP-xylose, low activity of the xylosyltransferases, processive manner of the xylosyltransferases, or competing reactions such as serine phosphorylation, acylation, or other forms of glycosylation.

After xylose addition, a linkage tetrasaccharide assembles by the transfer of two galactose residues catalyzed by unique members of the β1-4 galactosyl-, β1-3 galactosyl-, and β1-3 glucuronosyltransferase families of enzymes. This intermediate can undergo sulfation of the galactose residues and phosphorylation at the C-2 position of xylose. In general, sulfation and phosphorylation occur substoichiometrically. Galactose sulfation is found only in CS, but studies are ongoing to characterize its role in chain initiation, polymerization, and/or turnover.

After addition of the linkage tetrasaccharide, two types of reactions can occur: addition of β1-4GalNAc, resulting in initiation of chondroitin sulfate or addition of al-4GlcNAc, resulting in initiation of heparan sulfate. In chondroitin sulfate biosynthesis, chondroitin polymerizing factor (ChPF) and chondroitin synthases (CSS) add GlcA-GalNAc disaccharides to the nascent glycosaminoglycan chain. Biochemical evidence suggests that more than one Gal transferase may exist in vertebrates, but genetic studies in C. elegans suggest that a single enzyme (SQV5) is involved in N-acetylgalactosamine addition and chain polymerization. The glycosaminoglycan chain is modified by chondroitin sulfate transferases (e.g., chondroitin 4-O-sulfotransferase 1/CHST11, chondroitin 4-O-sulfotransferase 2/CHST12, chondroitin 4-O-sulfotransferase 3/CHST13, dermatan 4-O-sulfotransferase 1/CHST14, chondroitin 6-O-sulfotransferase 1/CHST3, uronyl 2-sulfotransferase/UST, carbohydrate (N-acetylgalactosamine 4-sulfate 6-O) sulfotransferase 15/CHST15), which also contribute to glycosaminoglycan chain heterogeneity. A list of sulfotransferases and their information is provided at Table 1 below.

TABLE 1

| | | | Gene | Protein | IUBMB* |
|---|---|---|---|---|---|
| | | | Accession | Accession | Enzyme |
| Symbol | Full Name | Other Names | Number | Number | Nomenclature |
| CHST11 | carbohydrate | Chondroitin 4- | NM_018413 | NP_060883 | EC 2.8.2.5 |
| | (chondroitin 4) | sulfotransferase 1, | | | |
| | sulfotransferase 11 | Chondroitin 4-O- | | | |
| | | sulfotransferase 1, | | | |
| | | chondroitin 4- | | | |
| | | sulfotransferase | | | |
| | | 11, C4ST1 | | | |
| CHST12 | carbohydrate | Chondroitin 4- | NM_018641 | NP_061111 | EC 2.8.2.5 |
| | (chondroitin 4) | sulfotransferase 2, | | | |
| | sulfotransferase 12 | Chondroitin 4-O- | | | |
| | | sulfotransferase 2, | | | |
| | | carbohydrate | | | |
| | | sulfotransferase | | | |
| | | 12, C4ST2 | | | |
| CHST13 | carbohydrate | Chondroitin 4- | NM_152889 | NP_690849 | EC 2.8.2.5 |
| | (chondroitin 4) | sulfotransferase 3, | | | |
| | sulfotransferase 13 | Chondroitin 4-O- | | | |
| | | sulfotransferase 3, | | | |
| | | carbohydrate | | | |
| | | sulfotransferase | | | |
| | | 13, C4ST3 | | | |
| CHST14 | carbohydrate (N- | dermatan 4 | NM_130468 | NP_569735 | EC 2.8.2.35 |
| | acetylgalactosamine | sulfotransferase 1, | | | |
| | 4-0) | carbohydrate | | | |
| | sulfotransferase 14 | sulfotransferase | | | |
| | | 14, D4ST1 | | | |
| CHST3 | carbohydrate | Chondroitin 6-O- | NM_004273 | NP_004264 | EC 2.8.2.17 |
| | (chondroitin 6) | sulfotransferase 1, | | | |
| | sulfotransferase 3 | carbohydrate | | | |
| | | sulfotransferase 3, | | | |
| | | C6ST1 | | | |
| CHST15 | carbohydrate (N- | N-acetylgalactosamine | NM_001270764 | NP_001257693 | EC 2.8.2.33 |
| | acetylgalactosamine | 4-sulfate 6-O- | | | |
| | 4-sulfate 6-O) | sulfotransferase, B | | | |
| | sulfotransferase 15 | cell RAG associated | | | |
| | | protein (GALNAC4S-6ST), | | | |
| | | carbohydrate | | | |
| | | sulfotransferase | | | |
| | | 15, GalNAc4S- | | | |
| | | 6ST, GALNAC4S6ST | | | |
| UST | uronyl-2- | dermatan/chondroitin | NM_005715 | NP_005706 | EC 2.8.2. |
| | sulfotransferase | sulfate 2- | | | |
| | | sulfotransferase, | | | |
| | | 2OST, DS2ST | | | |

*International Union of Biochemistry and Molecular Biology

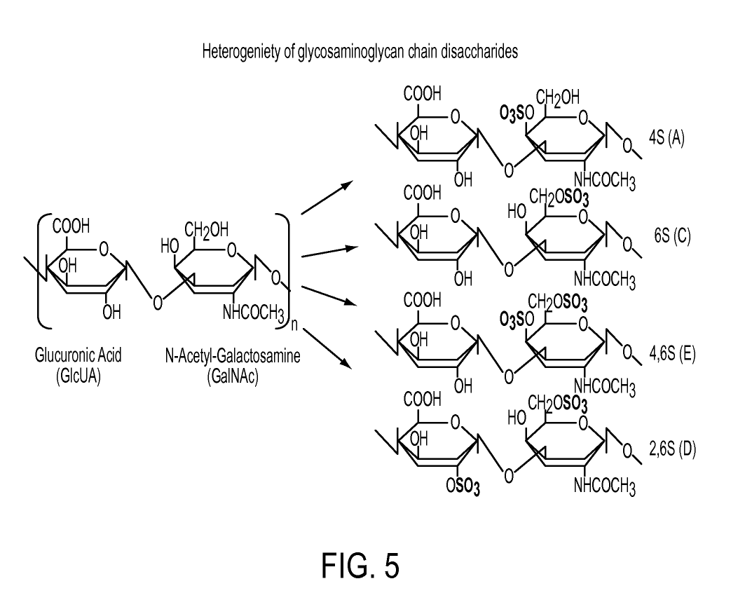

Chondroitinases exist with specificity for particular chondroitin sulfate substrates. Chondroitinase bacterial preparations have been characterized against different chondroitin sulfate (CS) substrates to yield a series of distinct chondroitinases: Chondroitinase AC that degrades mostly chondroitin sulfate A (CS-A) and chondroitin C (CS-C) (Yamagata et al., 1968), Chondroitinase B that degrades chondroitin B (CS-B) (Michelacci and Deitrich, 1976), Chondroitinase C that acts mostly on CS-C (Michelacci Y M & Deitrich C P, 1976) and Chondroitinase ABC exhibits specificity against all three substrates—CS-A, CS-B and CS-C (Yamagata et al., 1968, Michelacci et al., 1987). Glycosaminoglycan chain disaccharides are heterogeneous due in part to different types of sulfation of glucuronic acid (GlcA) and N-Acetylgalactosamine (GalNAc), as depicted in FIG. 5. Chondroitinases, which cleave at specific units—CS-A at A units, B at DS units, etc., will degrade most GAG chains, even though GAG chains are heterogeneous. In particular, the 4-sulfation of GalNAc has been shown to be important in the methods of the invention.

### Chondroitin/dermatan N-acetylgalactosamine-4-O-sulfotransferases (GalNAc 4-O-transferases)

Preventing, inhibiting, or reducing the biosynthesis of 4-sulfated chondroitin sulfate is useful in the methods of the invention Inhibiting chondroitin sulfate transferases (CHST11, 12, 13, 14, 15) (e.g., using an siRNA) blocks 4-sulfation of glycosaminglycan chains. In particular, inhibiting the activity or amount of chondroitin N-acetylgalactosamine-4-O-sulfotransferases is expected to reduce 4-sulfation of glycosaminoglycan chains, which have repellent activity on neuron regeneration. Exemplary GalNAc 4-O-transferases in humans that have been characterized include: dermatan 4-O-sulfotransferase-1/carbohydrate sulfotransferase 14 (D4ST1/CHST14) and chondroitin 4-O-sulfotransferases-1, -2, and -3 (C4ST1/CHST11, C4ST2/CHST12, and C4ST3/CHST13).

The amino acid sequence of dermatan 4-O-sulfotransferase-1/D4ST1/CHST14 is provided at NCBI Accession No. NP_{—}569735, which is reproduced below:

```
    1 mfprpltpla apngaeplgr alrraplgra raglggppll
        lpsmlmfavi vassglllmi
  61 ergilaemkp lplhppgreg tawrgkapkp gglslragda
        dlqvrqdvrn rtlravcgqp
121 gmprdpwdlp vgqrrtllrh ilvsdryrfl ycyvpkvacs
        nwkrvmkvla gvldsvdvrl
181 kmdhrsdlvf ladlrpeeir yrlqhyfkfl fvreplerll
        sayrnkfgei reyqqrygae
241 ivrryragag pspagddvtf peflrylvde dpermnehwm
        pvyhlcqpca vhydfvgsye
301 rleadanqvl ewvrapphvr fparqawyrp aspeslhyhl
        csaprallqd vlpkyildfs
361 lfayplpnvt keacqq
```

Dermatan 4-O-sulfotransferase-1 is a 376-amino acid polypeptide. D4ST1 has GalNAc 4-O-transferases activity and catalyzes the addition of C4 sulfate group to GalNAc. The polypeptide has a conserved GalNAc 4-O-transferase domain, including the active site, spanning residues 144-364. The nucleotide sequence of an mRNA transcript encoding dermatan 4-O-sulfotransferase-1/D4ST1/CHST14 corresponds to NCBI Accession No. NM_{—}130468 (human D4ST1 encoded at nucleotides 254-1384), which is reproduced below:

```
      1 ctgcgaggcc gggggtggag agcggccggg cgggacatcc ggcccgggtc cctcgccgcg
    61 cccgccgccc gccgcccgct tcggcgccgc agcccgggag ccggccaccc ctacacgcgc
  121 cagggctgtc ccctgccctc ccctccccaa ctacccccgg tcccagaccc tcctcccgcc
  181 cccagcccga gcccgccttc caggccgccc tcggatcggc cgggcccgcg caggccccca
  241 ccccttgagc accatgttcc cccgcccgct gaccccgctg gcggccccaa atggcgccga
  301 gcccctgggc cgggcgctga ggcgggcccc tctgggcagg gcccgggcgg ggctgggtgg
  361 gccgcccctg ctgctgccgt ccatgctgat gtttgcggtg atcgtggcct ccagcgggct
  421 gctgctcatg atcgagcggg gcatcctggc cgagatgaag cccctgcccc tgcacccgcc
  481 cggccgcgag ggcacagcct ggcgcgggaa agcccccaag cctgggggcc tgtccctcag
  541 ggctggggac gcggacttgc aagtgcggca ggacgtccgg aacaggaccc tgcgggcggt
  601 gtgcggacag ccaggcatgc cccgggaccc ctgggacttg ccggtggggc agcggcgcac
  661 cctgctgcgc cacatcctcg taagtgaccg ttaccgcttc ctctactgct acgtccccaa
  721 ggtggcctgc tctaactgga agcgggtgat gaaggtgctg gcaggcgtcc tggacagcgt
  781 ggacgtccgc ctcaagatgg accaccgcag tgacctggtg ttcctggccg acctgcggcc
  841 tgaggagatt cgctaccgcc tgcagcacta ctttaagttc ctgtttgtgc gggagccctt
  901 ggaacgcctc ctctctgcct accgcaacaa gtttggcgag atccgagagt accagcaacg
  961 ctatggggct gagatagtga ggcggtacag ggctggagcg gggcccagcc ctgcaggcga
1021 cgatgtcaca ttccccgagt tcctgagata cctggtggat gaggaccctg agcgcatgaa
1081 tgagcattgg atgcccgtgt accacctgtg ccagccttgt gccgtgcact atgactttgt
1141 gggctcctat gagaggctgg aggctgatgc aaatcaggtg ctggagtggg tacgggcacc
1201 acctcacgtc cgatttccag ctcgccaggc ctggtaccgg ccagccagcc ccgaaagcct
1261 gcattaccac ttgtgcagtg ccccccgggc cctgctgcag gatgtgctgc ctaagtatat
1321 cctggacttc tccctctttg cctacccact gcctaatgtc accaaggagg cgtgtcagca
1381 gtgaccatgg gtgtggggcc agcagctggt ggggactggt ttcaacgcca gctttctgtg
1441 cttctgcctg tcattcggag aaactctggc tctggggctt ggggcttctc aggatcctgg
1501 atggcagaga ctgccctcag aagttccttg tccagggtgg gcacccacag tgactcagag
1561 gacagggcta ggcaggagac ctgctgctcc tcattggggg gatctcttgg ggggcagaca
1621 ccagtttgcc aatgaagcaa cacatctgat ctaaagactg gctccagacc ccgggctgcc
1681 aggattatgc agtccacttg gtctacctta atttaacctg tggccaaact cagagatggt
1741 accagccagg ggcaagcatg accagagcca gggaccctgt ggctctgatc ccccatttat
1801 ccaccccatg tgcctcagga ctagagtgag caatcatacc ttataaatga cttttgtgcc
1861 tttctgctcc agtctcaaaa tttcctacac ctgccagttc tttacatttt tccaaggaaa
1921 ggaaaacgga agcagggttc ttgcctggta gctccaggac ccagctctgc aggcacccaa
1981 agaccctctg tgcccagcct cttccttgag ttctcggaac ctcctcccta attctccctt
2041 ccttccccac aaggcctttg aggttgtgac tgtggctggt atatctggct gccatttttc
2101 tgatgcattt atttaaaatt tgtacttttt gatagaaccc ttgtaagggc tttgttttcc
2161 taatagctga ctttttaata aagcagtttt atatataaaa aaaaaaaaaa aaa
```

The amino acid sequence of chondroitin 4-O-sulfotransferase-1/C4ST1/CHST11 is provided at NCBI Accession No. NP_{—}060883, which is reproduced below:

```
    1 mkpallevmr mnricrmvla tclgsfilvi fyfqsmlhpv
        mrrnpfgvdi ccrkgsrspl
  61 qelynpiqle lsntavlhqm rrdqvtdtcr ansatsrkrr
        vltpndlkhl vvdedheliy
121 cyvpkvactn wkrlmmvltg rgkysdpmei paneahvsan
        lktlnqysip einhrlksym
181 kflfvrepfe rlvsayrnkf tqkynisfhk rygtkiikrq
        rknatqealr kgddvkfeef
241 vaylidphtq reepfnehwq tvyslchpch ihydlvgkye
        tleedsnyvl qlagvgsylk
301 fptyakstrt tdemtteffq nissehqtql yevykldflm
        fnysvpsylk le
```

Chondroitin 4-O-sulfotransferase-1/C4ST1/CHST11 is a 352-amino acid polypeptide. C4ST1 has GalNAc 4-O-transferases activity and catalyzes the addition of C4 sulfate group to GalNAc. The polypeptide has a conserved GalNAc 4-O-transferase domain, including the active site, spanning residues 113-343. The nucleotide sequence of an mRNA transcript encoding chondroitin 4-O-sulfotransferase-1/C4ST1/CHST11 corresponds to NCBI Accession No. NM_{—}018413 (human C4ST1 encoded at nucleotides 499-1557), which is reproduced below:

```
      1 aggcgccctc ccctcgccgg gggccgcgag ttgcatttgg taaaacccag ccccggaata
    61 tatagatcgt tggagcgcaa tgaagtagcc tttggagaga agggagaggg cccgtcggac
  121 agccacagcg gccagcgcag cggcagcggc ggcggcacca ccatcaccgc tcgcacccca
  181 gccgcccggc ccgcgaccag gcagcggcgg ccgccggcgg gatcggagga ggcggcggag
  241 cggcgaggag gaggagcagg agcgcgcagc cagcgggtcc acgcatctca gcacttccag
  301 accaactccg gcaccttcca cacccctgcc cgggctgggg gctccgagag cggccgcgaa
  361 gcgactccga tcctccctct gagccttgct cagctctgcc ccgcgcctcc cgggctccgg
  421 tccgcgcggc ggggtccctg ctcctgcgcc ccgggcgcgc ttcccggaca ccccggtccc
  481 cgcagccagg acaaagccat gaagccagcg ctgctggaag tgatgaggat gaacagaatc
  541 tgccggatgg tgctggccac ttgcttggga tcctttatcc tggtcatctt ctatttccaa
  601 agtatgttgc acccagtcat gcggaggaat ccctttggtg tggacatctg ctgccggaag
  661 gggtcccgaa gccccctgca ggaactctac aacccaatcc agctggagct ctcaaacact
  721 gctgtcctgc accagatgcg gcgggaccag gtgacagaca cgtgccgagc caacagcgcc
  781 acaagccgta agcggagggt gctgaccccc aacgacctga agcacttggt ggtggatgag
  841 gaccacgagc tcatctactg ctacgtgccc aaggtggcct gcaccaactg gaagcggctc
  901 atgatggtcc tgaccgggcg ggggaagtac agcgacccca tggagatccc ggccaacgag
  961 gcacacgtct ccgccaacct gaagaccctg aaccagtaca gcatcccaga aatcaaccac
1021 cgcttgaaaa gctacatgaa gttcctgttt gtccgggagc ccttcgagag gctagtgtcc
1081 gcctaccgca acaagttcac ccagaagtac aacatctcct tccacaagcg gtacggcacc
1141 aagatcatca aacgccagcg gaagaacgcc acccaggagg ccctgcgcaa aggggacgat
1201 gtcaaattcg aggagtttgt ggcctatctc atcgacccac acacccagcg ggaggagcct
1261 ttcaacgaac actggcaaac cgtctactca ctctgccatc cctgccacat ccactatgac
1321 ctcgtgggca agtacgagac actggaagag gattctaatt acgtcctgca gctggcagga
1381 gtgggcagct acctgaagtt ccccacctat gcaaagtcta cgagaactac tgatgaaatg
1441 accacagaat tcttccagaa catcagctca gagcaccaaa cgcagctgta cgaagtctac
1501 aaactcgatt ttttaatgtt caattactca gtgccaagct acctgaaatt ggaataaagg
1561 gggtggggag agggagagaa tcatgctttt taatttaaga tttttatttg tcaaaagaat
1621 tatatggata ttgggttatt ttgtaaatta atatttcttt ggggatgatg ctgcgagcag
1681 catagtgaga attatttaaa atccttcgta gggaaggaca gctgtctttg caggggaaat
1741 aggatgggtc gtccttgtct gtagaagtga atactgcaac actgtctcaa aggtttcttg
1801 tgttctggtg aattccatga attgtgcatt ccataaattc taattaatat tatttatagt
1861 tatttaaaca tggtctcatt atttcttttt gtggcagcaa aattctaaca tctttccaga
1921 agaaatctat gattcctgct ttgcttgcta cagctcattt gggggcatga atgttctcct
1981 tactaacctc tcatggagtc atgtgataac agccattgtc atgtgtatgg atgccaccat
2041 tcttgaattt ctgctagaaa taagtaggtt gctattgcac ttagagtcat ctttgcaagg
2101 gactcattcc aaaccaattc caatcgacct gaaagtccct tgcactaaag aaatgtgatt
2161 tttttttaac ccaaggagaa agttggttgt aacaattttt caccagaatt agcaatctga
2221 tagaataggc ttttttaaca ggaattttaa actggcggtg ccagttgcag tgaaggtgaa
2281 gtggctattg ctttatattc gacaaatatt tattgagcat ctactatata ctaggccctg
2341 aagatagagc gttgaacaaa cccaatagtc ttggccctca aaaagcttta tgtgacatag
2401 agcatccagc tcgacaatta gcaaacccct gatattattc atggacttga catgaggttt
2461 ggactattca tgagcaacag cagaaggtcc atgaccccta caaatttaca tttttctatg
2521 gcccatattt gaaaacctca cattcggagc aggccacttt atattcgtga gtgatcccaa
2581 gctcactgac caacagaaaa gatgctaaaa aaatgtgtcc tatcatgtgc ctgctccaac
2641 tgatgggtaa tgtctgcctt taagccccag aatggattct ccaggcacag tgtggaagag
2701 tgctgtggtt gattagcaat gtctgccttg ggtgaggaaa ggggaacact cctcaagtgc
2761 ctcctactgt catctccatt cagcctagca ccacatcccc atgtagaagt gttgtcttct
2821 actgcttgcc acgtacaagt gtgaaatgcc aaggtgagac tcgtacactt ggggatttgc
2881 aatggttttt acccctcatg aaggtcagag gtggactatt tgtacacata cacacatgca
2941 caccctctca cgtcctccag aggctgaaag actatcagca tcttggtaac tgccatgcat
3001 gagcagcaag gacagccttc cataatccat gccccatttc ctctggaagc ctggaaactt
3061 ttcatttcct gttccttgct aattaacatc taaacatgct tctcatttcc agccattgct
3121 gatgcttctc agttgaagtt tgagccacat ccctcttaca gctagtgaat gagttggtag
3181 cagctactgt atataataat aataatagtt ttaataatag gggagggtgg gatggggtgt
3241 gggtaagttt tgccttttgt tttgtttttg atgccgtata tagtgaaggg gtgaggatat
3301 tctaaacaaa caaaaattga ataatttatt cacagaaact attaagattg tattgtaaag
3361 ctcacagcaa gctcagtggg cagcagctgc aacatctcac cggggaaatt attttattta
3421 acgtgagtga gatgtgggcc ggagaaggta gctgaagcta tttataaaac gttgtgtacc
3481 ttcttccgag ctctctcccc tttgtgaagg gcgcagcaac tatacccttg atggatggag
3541 atttacgcaa tgtgttttac tgggtagagt gacagacctt ggctgtccct ggaattgaga
3601 atctggacct tatttccagg cagagaacac tgtcctcaga aatgggactt ctgataatga
3661 aacaggttgt ccaacatttt ctaatacgca ttctacagaa cacaggttcc aggaggcatc
3721 aggttggtgt gatgcagact aagggttttg tggtcaaata tccttaagaa acaaagttaa
3781 gtcagtttcc tttctgcaag gacttttcag ccttcaatgt gtgctgtgca tggtgaatcc
3841 ccaaaaataa tccttgagtc tgtagcattt cctgaactta catgaccagg atactctatt
3901 ttgcagatca ccccacagag ctagggttcc accgagtata ctttgcttag gttgacttag
3961 catatctgag gtcttcaagg ataaaaactg ccaccccaac acccttccat taaaaaaaaa
4021 atacaaaata gcaccacagt ctccatctgg tttatagcaa cagaggtact ttatttaatg
4081 aagcaatggt tctaatcctg gatactgcca cggactacaa ttctatccct cccagaggga
4141 gtggaggaag tcttgggtgg tgtggacaga aggaagagag gagagggtga gtggggtata
4201 gggcccaggg tggctcccta ctcctcaggc tcaaaaggat gctcagtggg aacagatgat
4261 ctcttgatga gtgcttcttc agtttcatag tttggaatcg ttcactgtgt gctttttggg
4321 gggttttcaa tggaaattca cgttgctttg catttctgtg tccgtctttg gtcagttgtg
4381 caagcctgct cactgtcatg tgaagatggc ctttcatctg gcttctctct cttaagtgag
4441 aaagattgtc cttcagggga catgacatca ataggtttct ggaatgaggg actctttctc
4501 cccgtgtttt gctttgtgtt cacattttct tttctaatgg cattgaaact ttaaaaaaaa
4561 tggattcaac tgtttttgca gaatgtagaa agtattctgt gtccttggtt aaagaaatcc
4621 acttgtgaag tgtgcctgga aaatgaaagt ttgtgttttt taaagaggaa tatttgaaac
4681 tgctttctat gcatgcttag ctggagaaaa gtacaggcag gcgtcccatc tcccagccac
4741 ttctcaaagg tgctgctgtg ttttaaagac caggtacagc cagggcagta tttgcaagga
4801 cattcctgct tactttatcc ctttggttgg aaagctctag atgattcccg cagctcctcc
4861 agaccccgcc tccctgccct ccccagctgg tctgggaaga ggtggtctgc tgacctgtgg
4921 tatctcagag gggacgttcc tcctcctccc tgtgcaccag gtgggctgca ccctcctgcc
4981 tattcaggat gtggatgcca caggagagca gcaggcagtg gaaacttcag ttgcactggt
5041 tctcctggtg gcaaaggcat gaagcacagg ggtcgattaa tccaggctac tagaaagctc
5101 cagagcaaag tgtgcgggtc ccacaaatgc ttggctggtg gggtctggat cagtgctgag
5161 atagagttgg cagaagaagc agaggcactc tgcttgcttt cctagccagt cctcccctac
5221 acacacacac acacacacac acacacacac acacaatctc agctgcgcca ttctgtgcaa
5281 tcccagtgac caaatccctt ccttgcccac ctctatgtca gcaggactga ccacatcact
5341 cccccgagtt cccaccacca gcatttcctc caaccttttt ccatcacaac cagttagaac
5401 cctacaggca acaaggcctt ctagaatccg cttaaccctt ggctgataac aggcaaattt
5461 cagtctgcta cactttgtta ggtccagaag gagctgccca tactactttc ttatgagcat
5521 gctcagtatg gcatatggac atgtaatgtc acatctttgt ggagtgtgat tttctttttt
5581 cacatatttg tatgcagtag agagcctgtt gtagaaaacg ctccctgtat cttgctgtac
5641 tgttaaagaa agctgaattc cacattgcca acaaaagcgt gaaaatgttc atgaaccttc
5701 ctccaggaaa agccattcaa gcctgattat ttttctaagt aacttcaatt aaattgaaga
5761 aaaaagaa
```

The amino acid sequence of chondroitin 4-O-sulfotransferase-2/C4ST2/CHST12 is provided at NCBI Accession No. NP_{—}061111, which is reproduced below:

```
    1 mtkarlfrlw lvlgsvfmil liivywdsag aahfylhtsf
        srphtgpplp tpgpdrdrel
  61 tadsdvdefl dkflsagvkq sdlprketeq ppapgsmees
        vrgydwsprd arrspdqgrq
121 qaerrsvlrg fcansslafp tkerafddip nselshlivd
        drhgaiycyv pkvactnwkr
181 vmivlsgsll hrgapyrdpl riprehvhna sahltfnkfw
        rrygklsrhl mkvklkkytk
241 flfvrdpfvr lisafrskfe leneefyrkf avpmlrlyan
        htslpasare afraglkvsf
301 anfiqylldp hteklapfne hwrqvyrlch pcqidydfvg
        kletldedaa qllqllqvdr
361 qlrfppsyrn rtassweedw fakiplawrq qlyklyeadf
        vlfgypkpen llrd
```

Chondroitin 4-O-sulfotransferase-2 is a 414-amino acid polypeptide. C4ST-2 has GalNAc 4-O-transferases activity and catalyzes the addition of C4 sulfate group to GalNAc. The polypeptide has a conserved GalNAc 4-O-transferase domain, including the active site, spanning residues 160-405. The nucleotide sequence of an mRNA transcript encoding chondroitin 4-O-sulfotransferase-2/C4ST2/CHST12 corresponds to NCBI Accession No. NM_{—}001243794 (human C4ST-2 encoded at nucleotides 164-1408), which is reproduced below:

```
      1 gctcctgcgc cgggggcggg gccggcgagg gctgcctctg gggggtccgc tagtcgcggg
    61 gcggcggcgg cggctgcggg cgcgaggttc ccagcaggat gccccggctc tgcaggaagc
  121 tgaagtgaga ggcccggaga gggcccagcc cgcccggggc aggatgacca aggcccggct
  181 gttccggctg tggctggtgc tggggtcggt gttcatgatc ctgctgatca tcgtgtactg
  241 ggacagcgca ggcgccgcgc acttctactt gcacacgtcc ttctctaggc cgcacacggg
  301 gccgccgctg cccacgcccg ggccggacag ggacagggag ctcacggccg actccgatgt
  361 cgacgagttt ctggacaagt ttctcagtgc tggcgtgaag cagagcgacc ttcccagaaa
  421 ggagacggag cagccgcctg cgccggggag catggaggag agcgtgagag gctacgactg
  481 gtccccgcgc gacgcccggc gcagcccaga ccagggccgg cagcaggcgg agcggaggag
  541 cgtgctgcgg ggcttctgcg ccaactccag cctggccttc cccaccaagg agcgcgcatt
  601 cgacgacatc cccaactcgg agctgagcca cctgatcgtg gacgaccggc acggggccat
  661 ctactgctac gtgcccaagg tggcctgcac caactggaag cgcgtgatga tcgtgctgag
  721 cggaagcctg ctgcaccgcg gtgcgcccta ccgcgacccg ctgcgcatcc cgcgcgagca
  781 cgtgcacaac gccagcgcgc acctgacctt caacaagttc tggcgccgct acgggaagct
  841 ctcccgccac ctcatgaagg tcaagctcaa gaagtacacc aagttcctct tcgtgcgcga
  901 ccccttcgtg cgcctgatct ccgccttccg cagcaagttc gagctggaga acgaggagtt
  961 ctaccgcaag ttcgccgtgc ccatgctgcg gctgtacgcc aaccacacca gcctgcccgc
1021 ctcggcgcgc gaggccttcc gcgctggcct caaggtgtcc ttcgccaact tcatccagta
1081 cctgctggac ccgcacacgg agaagctggc gcccttcaac gagcactggc ggcaggtgta
1141 ccgcctctgc cacccgtgcc agatcgacta cgacttcgtg gggaagctgg agactctgga
1201 cgaggacgcc gcgcagctgc tgcagctact ccaggtggac cggcagctcc gcttcccccc
1261 gagctaccgg aacaggaccg ccagcagctg ggaggaggac tggttcgcca agatccccct
1321 ggcctggagg cagcagctgt ataaactcta cgaggccgac tttgttctct tcggctaccc
1381 caagcccgaa aacctcctcc gagactgaaa gctttcgcgt tgctttttct cgcgtgcctg
1441 gaacctgacg cacgcgcact ccagtttttt tatgacctac gattttgcaa tctgggcttc
1501 ttgttcactc cactgcctct atccattgag tactgtatcg atattgtttt ttaagattaa
1561 tatatttcag gtatttaata cgaaatgtgg aagggaatgc tggagtaaaa tatcccctct
1621 cccctccgcc cgcccacccg cccgcccgct cgcccgctcg cccgctcctg tggtttttct
1681 gagcgtgcgg gcgccgggag gggatgctga ggctgatgga gctgcctcca gggctagggc
1741 cactcaccgg aggagggcgg ggcctgcact tgaagtcagg ccgcacctgt ctgtttttgg
1801 aagggtagcc gacaaatcct tccagaggga aagttctttg tttaagtgtt gtacttgaaa
1861 aggtcaatct tcagggcttc ctgtttgaag tcaagtcaga ggtaaaccgg tcagttacag
1921 aagcaggatt tctaggattt ctaactccag ctgttcccat actgtctagt ttaaattatg
1981 gctgttaagg ccgggcgggt gactcaggca ggtaatccca gaactttggg aggcccagac
2041 agaaggatcg cttgaggtca ggaatttgag acctggccaa catggtgaaa ccctgtctct
2101 actaaaaaaa aaaaaaaaaa aaaaaaaaaa a
```

The amino acid sequence of chondroitin 4-O-sulfotransferase-3/C4ST3/CHST13 is provided at NCBI Accession No. NP_{—}690849, which is reproduced below:

```
    1 mgrrccrrrv laaaclgaal lllcaaprsl rpafgnralg
        sswlggekrs plqklydldq
  61 dprstlakvh rqrrdllnsa csrhsrrqrl lqpedlrhvl
        vddahgllyc yvpkvactnw
121 krvllalsgq argdpraisa qeahapgrlp sladfspaei
        nrrlraylaf lfvrepferl
181 asayrnklar pysaafqrry garivqrlrp ralpdararg
        hdvrfaefla ylldprtrre
241 epfnehwera halchpcrlr ydvvgkfetl aedaafvlgl
        agasdlsfpg pprprgaaas
301 rdlaarlfrd ispfyqrrlf dlykmdfllf nysapsylrl
        l
```

Chondroitin 4-O-sulfotransferase-3 is a 341-amino acid polypeptide. C4ST-3 has GalNAc 4-O-transferases activity and catalyzes the addition of C4 sulfate group to GalNAc. The polypeptide has a conserved GalNAc 4-O-transferase domain, including the active site, spanning residues 102-332. The nucleotide sequence of an mRNA transcript encoding chondroitin 4-O-sulfotransferase-3/C4ST3/CHST13 corresponds to NCBI Accession No. NM_{—}152889 (human C4ST-3 encoded at nucleotides 46-1071), which is reproduced below:

```
      1 cagccgtatc cagcggactg tcctccgccg cgcgcccggc acagcatggg gaggcgctgc
    61 tgccggcggc gcgtgctggc ggccgcctgt ctgggcgccg cgctcctgct cctatgcgcc
  121 gcgccccgct ccctgcgccc ggcatttgga aacagagccc tgggctccag ctggcttggt
  181 ggggagaaga gaagccccct gcagaagctc tatgacctgg atcaggaccc gcgctcgacc
  241 ctggcgaagg tgcaccggca gcggcgcgac ctgctgaaca gcgcctgtag ccgccactca
  301 cgccggcagc gcctgctaca gccggaggac ctgcggcacg tgctggtgga cgacgcgcat
  361 ggcctgctct actgctacgt gcccaaggtg gcctgcacca actggaagcg cgtgctgctg
  421 gcgctgagcg gccaagcccg cggcgacccg cgcgccatct ccgcgcaaga ggcgcacgcg
  481 cctggccgcc tgccctcact ggccgacttc agccccgccg agatcaaccg gcgcctgcgc
  541 gcctacttgg ccttcctgtt cgtgcgggag cccttcgagc gcctggcatc ggcttaccgc
  601 aacaagctcg cgcgccccta cagcgccgcc ttccagaggc gctacggtgc acgcatcgtt
  661 cagcgcctgc ggccgcgcgc gctccccgac gcccgggccc gcggccacga cgtgcgcttc
  721 gcggagttcc tggcctacct gctggacccg cgcacgcggc gtgaggagcc cttcaacgag
  781 cactgggagc gcgcgcacgc gctctgccac ccgtgtcgcc tccgctacga cgtcgtgggc
  841 aagttcgaga cgctggcgga ggacgcggcc ttcgtgctgg gcctggcggg cgcatccgac
  901 ctgagcttcc ctgggccgcc gcggccccgg ggagccgccg cctcccgcga cctggcagcg
  961 cgcctcttcc gggacatcag ccccttctac cagcggcgcc tcttcgacct ctacaagatg
1021 gacttcctgc ttttcaacta ctccgccccc tcctacctgc ggctgctcta gcggtcctgg
1081 aggtcctgtg gccacgcggg gcaagtgcct ttccgacaag acccccgggg aatgcaggtg
1141 ctgccggccc caggacccct cttcaagagc cactgcgtgc actcacctgg ccgccgggcc
1201 agcgggcgca gggcacacct ggccaggctt gggggcagcc catctcaggt ggccctgcac
1261 gcgtgtgcct gcctcggcct gtcgcctgag gcctgcttcc tccacttgct ccagctgaca
1321 ggcacctctc caggccccgt agatgggcaa ggacttgata accagggttt taggctttta
1381 aaggccattt tgggggtcag ccctgcccct gaacctgttc atggtgcatc agaacataat
1441 gctgacaccg gtgtcagtgt ggcccgagcc tgtgtcctcc ccacctcgcc caccctggca
1501 aggacagctg cggccaagga cgaaagccct cccttggctg gcctcacgat ggggccgtcc
1561 cgggagccag gtgggagctg ccttccactg ccatcgggtc tcctctcctc tcccacgcgg
1621 ctggccctac ccaggcgcca ccttcggtct cagtctggca agacgctggg tcttcaggct
1681 ccatgccaac agagcccctg gtgcaatgcg gtcacaggtt ttatgggact ttggtgagct
1741 gggcggtcat ggttttgaaa taaatgtatt ttgttacttt ctgaaaaaaa aa
```

Arylsulfatase B (ARSB; GalNAc 4-sulfatase)

Arylsulfatase B (ARSB; GalNAc 4-sulfatase) is a N-acetylgalactosamine (GalNAc) 4-sulfatase (Enzyme Nomenclature Number 3.1.6.12). ARSB localizes to the lysosome where it catalyzes the removal of C4 sulfate group from GalNAc at the nonreducing terminus of chondroitin sulfate (CS)/dermatan sulfate (DS) gylcosylaminoglycan (GAG) chains during lysosomal degradation.

Mucopolysaccharidosis (MPS) VI is an autosomal recessive lysosomal storage disorder resulting from a deficiency of ARSB, typically due to a mutation in the gene encoding the enzyme. Clinical features and severity of MPS VI are variable, but usually include short stature, hepatosplenomegaly, dysostosis multiplex, stiff joints, corneal clouding, cardiac abnormalities, and facial dysmorphism. Intelligence of MPS VI patients is usually normal. ARSB is clinically approved for use in humans for the treatment of MPS VI. In contrast to therapies involving chondroitinase ABC (cABC), ARSB has a narrower target than cABC (i.e., 4-sulfation at non-reducing ends of CS-GAGs).

The amino acid sequence of human Arylsulfatase B (ARSB) is provided at NCBI Accession No. NP_{—}000037, which is reproduced below:

```
    1 mgprgaaslp rgpgprrlll pvvlplllll llappgsgag
        asrpphlvfl laddlgwndv
  61 gfhgsrirtp hldalaaggv lldnyytqpl ctpsrsqllt
        gryqirtglq hqiiwpcqps
121 cvpldekllp qllkeagytt hmvgkwhlgm yrkeclptrr
        gfdtyfgyll gsedyysher
181 ctlidalnvt rcaldfrdge evatgyknmy stniftkrai
        alitnhppek plflylalqs
241 vheplqvpee ylkpydfiqd knrhhyagmv slmdeavgnv
        taalkssglw nntvfifstd
301 nggqtlaggn nwplrgrkws lweggvrgvg fvaspllkqk
        gvknrelihi sdwlptivkl
361 arghtngtkp ldgfdvwkti segspsprie llhnidpnfv
        dsspcprnsm apakddsslp
421 eysafntsvh aairhgnwkl ltgypgcgyw fpppsqynvs
        eipssdpptk tlwlfdidrd
481 peerhdlsre yphivtklls rlqfyhkhsv pvyfpaqdpr
        cdpkatgvwg pwm
```

Human ARSB is a 533-amino acid polypeptide. ARSB has N-acetylgalactosamine (GalNAc) 4-sulfatase activity and catalyzes the removal of C4 sulfate group from GalNAc at the nonreducing terminus of chondroitin sulfate (CS)/dermatan sulfate (DS) gylcosylaminoglycan (GAG) chains. Calcium ion is a co-factor for ARSB 4-sulfatase activity. Identification of important active site residues and characterization of their roles include: Asp53 (Ligand of metal ion); Asp54 (Ligand of metal ion); Cys91 (Nucleophile, forms covalent sulfoenzyme intermediate); Cys91 (Ligand of metal ion); His147 (General base, activates nucleophile cysteine residue C); Asp300 (Ligand of metal ion); Asn301 (Ligand of metal ion). The nucleotide sequence of an mRNA transcript encoding human arylsulfatase B corresponds to NCBI Accession No. NM_{—}000046 (human arylsulfatase B encoded at nucleotides 1287-2888), which is reproduced below:

```
      1 aaaagtgaat acatgatttt atttaactca ttaataagga aattggtaag gtgttaaaac
    61 caattcaaag gacaatccaa agaacagatc aggaatacta aaataaatat gcaagcggag
  121 gtgaaactgt tttccttggt agtggtggag gggaaggatt gctactccgc tggataaagt
  181 tcatttgtgt atatataaat aagaattatt ttccattgtt atttatctat aacttataaa
  241 gttgtaaaca acttccacgg aatcagactc aacctggaag ggtatggtct ctaggcaatg
  301 caaaaatttt cccctacacc tgttaacaac tataatatct ccagacagag tagacagaaa
  361 gtctggatgg caacgggaat ctactggtca tacggctaac ttcctaattc aataagcacg
  421 tgactaaagg attttttcct tccactcaga tatttcaggc taactagata ctgtgtgctt
  481 cttagtgtca ctgcttagtg ggggagccag ctctgagtgg ggtcatatcc ggacaagcga
  541 atgagctatt tattcaatga ccacgcaaca ctccaaatcc tcccagggca acttgaaagt
  601 aaccgcacct tccaaagggc accgtgcaat cagactgtgt gtttggcctc ctgtttgcta
  661 gtggggagga agcggcttca tgggtgtaca ctacgcataa atgaatgtga aaggctattt
  721 agacctctgc cttttcaccg tcctcccacc tgccacaggc tgggctcttg tgctagaaat
  781 gacttgctag ctagacatca tggttcagga tctgagtcag aggtttaacc atttataagc
  841 ttttttctta tgaaaaattg gcactaatta taatgtctaa ctgtcagagt tgttgcaggc
  901 tttacaggag acgcgggctg tgaagatgct ttgtaaattg tgaagcgtta ttaaagaaca
  961 catctttttt ttttaggaaa ccacagtgca aatttaattg ccggggaaga taacgggcct
1021 tggtgccctc caagcgtcag ctgagtttcc aagaagccgg gcagcgggcg cccgcgggtt
1081 cgtctctggc tcctcctccg ccacagcagc cgggggcccg ggtcggaggc ggcgggggcc
1141 gagcgcccgg cctcgcaagc ccacggcccg ctgggggtgc cgtcccgcgc cggggcggag
1201 caggccccgg cagcccagtt cctcattcta tcagcggtac aaggggctgg tggcgccaca
1261 ggcgctggga ccgcgggcgg acaaggatgg gtccgcgcgg cgcggcgagc ttgccccgag
1321 gccccggacc tcggcggctg ctcctccccg tcgtcctccc gctgctgctg ctgctgttgt
1381 tggcgccgcc gggctcgggc gccggggcca gccggccgcc ccacctggtc ttcttgctgg
1441 cagacgacct aggctggaac gacgtcggct tccacggctc ccgcatccgc acgccgcacc
1501 tggacgcgct ggcggccggc ggggtgctcc tggacaacta ctacacgcag ccgctgtgca
1561 cgccgtcgcg gagccagctg ctcactggcc gctaccagat ccgtacaggt ttacagcacc
1621 aaataatctg gccctgtcag cccagctgtg ttcctctgga tgaaaaactc ctgccccagc
1681 tcctaaaaga agcaggttat actacccata tggtcggaaa atggcacctg ggaatgtacc
1741 ggaaagaatg ccttccaacc cgccgaggat ttgataccta ctttggatat ctcctgggta
1801 gtgaagatta ttattcccat gaacgctgta cattaattga cgctctgaat gtcacacgat
1861 gtgctcttga ttttcgagat ggcgaagaag ttgcaacagg atataaaaat atgtattcaa
1921 caaacatatt caccaaaagg gctatagccc tcataactaa ccatccacca gagaagcctc
1981 tgtttctcta ccttgctctc cagtctgtgc atgagcccct tcaggtccct gaggaatact
2041 tgaagccata tgactttatc caagacaaga acaggcatca ctatgcagga atggtgtccc
2101 ttatggatga agcagtagga aatgtcactg cagctttaaa aagcagtggg ctctggaaca
2161 acacggtgtt catcttttct acagataacg gagggcagac tttggcaggg ggtaataact
2221 ggccccttcg aggaagaaaa tggagcctgt gggaaggagg cgtccgaggg gtgggctttg
2281 tggcaagccc cttgctgaag cagaagggcg tgaagaaccg ggagctcatc cacatctctg
2341 actggctgcc aacactcgtg aagctggcca ggggacacac caatggcaca aagcctctgg
2401 atggcttcga cgtgtggaaa accatcagtg aaggaagccc atcccccaga attgagctgc
2461 tgcataatat tgacccgaac ttcgtggact cttcaccgtg tcccaggaac agcatggctc
2521 cagcaaagga tgactcttct cttccagaat attcagcctt taacacatct gtccatgctg
2581 caattagaca tggaaattgg aaactcctca cgggctaccc aggctgtggt tactggttcc
2641 ctccaccgtc tcaatacaat gtttctgaga taccctcatc agacccacca accaagaccc
2701 tctggctctt tgatattgat cgggaccctg aagaaagaca tgacctgtcc agagaatatc
2761 ctcacatcgt cacaaagctc ctgtcccgcc tacagttcta ccataaacac tcagtccccg
2821 tgtacttccc tgcacaggac ccccgctgtg atcccaaggc cactggggtg tggggccctt
2881 ggatgtagga tttcagggag gctagaaaac ctttcaattg gaagttggac ctcaggcctt
2941 ttctcacgac tcttgtctca tttgttatcc caacctgggt tcacttggcc cttctcttgc
3001 tcttaaacca caccgaggtg tctaatttca acccctaatg catttaagaa gctgataaaa
3061 tctgcaacac tcctgctgtt ggctggagca tgtgtctaga ggtgggggtg gctgggttta
3121 tccccctttc ctaagccttg ggacagctgg gaacttaact tgaaatagga agttctcact
3181 gaatcctgga ggctggaaca gctggctctt ttagactcac aagtcagacg ttcgattccc
3241 ctctgccaat agccagtttt attggagtga atcacatttc ttacgcaaat gaagggagca
3301 gacagtgatt aatggttctg ttggccaagg cttctccctg tcggtgaagg atcatgttca
3361 ggcactccaa gtgaaccacc cctcttggtt caccccttac tcacttatct catcacagag
3421 cataaggccc attttgttgt tcaggtcaac agcaaaatgc ctgcaccatg actgtggctt
3481 ttaaaataaa gaaatgtgtt tttatcgtaa tttatttccc cccagccatt gctcactctg
3541 tctagacttc ctgccacttc caattcttct gtggcttttc ctgcctttcc ttttgacctc
3601 agtagtccta tccctgggaa ggccactttg cttctctacc tgagcacccc tgatttctgg
3661 aacgctgctg agccctgcct tacttttgcc cctagggctg aagctagagg cctccccgta
3721 ataggcggtg gagttgctct gtgaggatgt tcatggtaga cactaagagg gctgggtggg
3781 agatgcttgg ctctgtggca tctgttcagc gaggcttttc ctatattgca tggagttagt
3841 cattgtgatt gtagctttat ttcataatat attaagactt gcactgctat ttactagcag
3901 tgagaagaaa cctcaggaaa ggatatgaaa aagcaagtgg ccagtgtctg ggatactggg
3961 ccttggtaaa gcagaggagg gcacacccac agtcctctta ttctctgttt tactgcttgt
4021 tttgaggttc tggggtctgg caaagaggat gcagtttgac acctgcagcc ctttctcaat
4081 cccactaatg tcttactaat gtggaacagt ccatattagc tccagagagt gtcaaaccca
4141 gagaaatgtg tgcaaaaatg atactctttt ctgcattagc cccaccattg tgttcaccaa
4201 tgcttggaac actgcctgaa ggcactcatt ttttaatttt tattttattt ttaatttttt
4261 atatctttat gagacgatct cactctgtca ccaggttgga gtacagtggt acaatcacaa
4321 ctcaccgtag cctcaaactc ctgggctcaa gtgattctcc cacctcaggc acccaaatag
4381 ctggaactac aggcatatac cgccacaccc agctaatttt attttttgaa aagacaaggt
4441 tccctatgtt gcccagctgg tcttaaactc ctgggctcca gcaattatcc cagcttgggc
4501 tccaaaagtg ctgggattac aggcatgagt caccatgcct ggcctcattt tttaaaacaa
4561 atgaataaat ggacaaatga gtaaatgaga aagtctcaca ccatgaaaga tgctagtcca
4621 atgagctgaa tacagaggta atataaatgt cttccagctg ttgcttttct gttctcaagc
4681 tgcccctcct ggggtaggag cataatctac atcactgggc agtcacagga cactctatag
4741 caaggttgta gcgtcctctc cagtgggggg agaaaaggaa ctgtgcctac caaaggtact
4801 ctcttgtcag caatttccat ttctatactt tatgggacac tagaaactaa aagcaacaaa
4861 taatctgata taagtccttg tatagtcatc cttcaattca gtagcaatat tttctggtca
4921 ctactaacct gtattgtatt aaaatgagac tattggaagg aaatggtgct aaaactaata
4981 acatctctta ccaaccttta cccaactcct gggttggcaa acagctgacc aaactgccat
5041 cacctcccac ttggaagtgt atggccgaca gcatgaaata gctgagccca gatgttcctt
5101 ctgcatcctc cgaatcccag ggctgggtgt aggtagccgt tggaggccat cgctacaggg
5161 cacctatctg ttatcgctgc tgtcctccca acagctgtct ccagttctag ttccttggtt
5221 ttcaggcaca gtgggggatg ttctgcaccc agtggacttc aaaagagttt tgaagactta
5281 attttttgta aaacaagtac ttgagatttt ggtttatcca taatagaatg tatttcatta
5341 gattctctga ttctatataa gaatgtgaaa agattgatat attgttgtta gaaataatgt
5401 tatttctttc caattttttt tttttttttt tttgagatgg agtctcgctc tgtcacccag
5461 gctggagtgc agtggtgtga tctcggctca ctgcagcctc taactcccag gttcaagcta
5521 ttctcctgcc tcagcctccc aagtagctgg attacaggca tacaccacca cgcctggcta
5581 tgttttgtat ttttcgtaga gatagggttt caccatgttg gccaggctgg tctcaaactc
5641 ctgacctcaa gtgatccacc cacttcagct tcccaaagca ctgggattac aggtgtgagc
5701 cactgtgccc ggcaaatttt tttaccttta cagaaggttt tgcttattta attgtgagct
5761 catttttctt tgttactttt gtccccccag atttggggga caaaataaaa ttaatctttt
5821 aaaatgtgtc agccatatgt atggggcttc catttggggt gaggagaaag ttctggaact
5881 agatagtggt catggttata caacatcata aatgcaatta ctgccactga attgtatgtt
5941 ttaaagtggt taaaatgtta agttttatgt tttattacaa tttttaaatg tgtcaaccaa
6001 ctttatagta cataaattat atctcagtaa agctgttaaa taaataaata tagtaaaaat
6061 tttagaacta aaaaaa
```

### Stroke

A stroke, or cerebrovascular accident (CVA), is the rapid loss of brain function(s) due to disturbance in the blood supply to the brain. This can be due to ischemia (lack of blood flow) caused by blockage (thrombosis, arterial embolism), or a hemorrhage (leakage of blood). As a result, the affected area of the brain cannot function, which might result in an inability to move one or more limbs on one side of the body, inability to understand or formulate speech, or an inability to see one side of the visual field.

A stroke is a medical emergency and can cause permanent neurological damage, complications, and death. It is the leading cause of adult disability in the United States and Europe and the second leading cause of death worldwide. Risk factors for stroke include old age, hypertension (high blood pressure), previous stroke or transient ischemic attack (TIA), diabetes, high cholesterol, cigarette smoking and atrial fibrillation. High blood pressure is the most important modifiable risk factor of stroke.

An ischemic stroke is occasionally treated in a hospital with thrombolysis (also known as a “clot buster”), and some hemorrhagic strokes benefit from neurosurgery. Treatment to recover any lost function is termed stroke rehabilitation, ideally in a stroke unit and involving health professions such as speech and language therapy, physical therapy and occupational therapy. Prevention of recurrence may involve the administration of antiplatelet drugs such as aspirin and dipyridamole, control and reduction of hypertension, and the use of statins. Selected patients may benefit from carotid endarterectomy and the use of anticoagulants

Often times, a stroke seriously debilitates the patient. However, in those patients that due regain some brain function in affected areas, down-regulations of CSPGs are shown to occur. After stroke, plasticity occurs in some regions of the brain and is associated with some return of brain function. Rats that were able to recover from induced stroke had down-regulations of several CSPGs, including aggrecan, versican, and phosphacan. Rats that did not return any brain function did not have significant down-regulation of CSPGs. The reduction of CSPGs in rats that returned some brain function after stroke suggest that more neurological connections could be made with less CSPGs present. Medications that are able to down-regulate CSPGs may help return more brain function to stroke patients.

### Myocardial Infarction (MI)

Myocardial infarction (MI) or acute myocardial infarction (AMI), commonly known as a heart attack, results from the interruption of blood supply to a part of the heart, causing heart cells to die. This is most commonly due to occlusion (blockage) of a coronary artery following the rupture of a vulnerable atherosclerotic plaque, which is an unstable collection of lipids (cholesterol and fatty acids) and white blood cells (especially macrophages) in the wall of an artery. The resulting ischemia (restriction in blood supply) and ensuing oxygen shortage, if left untreated for a sufficient period of time, can cause damage or death (infarction) of heart muscle tissue (myocardium). There is data that indicates proteoglycans are upregulated after MI in the heart. Thus, proteoglycan upregulation has the potential to inhibit or prevent reinnervation of the heart. Accordingly, reducing 4-sulfation of glycosylaminoglycan chains or increasing removal of 4-sulfate from glycosylaminoglycan chains is useful for enhancing nerve regrowth in the heart following myocardial infarction.

Typical symptoms of acute myocardial infarction include sudden chest pain (typically radiating to the left arm or left side of the neck), shortness of breath, nausea, vomiting, palpitations, sweating, and anxiety (often described as a sense of impending doom). Women may experience fewer typical symptoms than men, most commonly shortness of breath, weakness, a feeling of indigestion, and fatigue. A sizeable proportion of myocardial infarctions (22-64%) are “silent”, that is without chest pain or other symptoms.

Among the diagnostic tests available to detect heart muscle damage are an electrocardiogram (ECG), echocardiography, cardiac MRI and various blood tests. The most often used blood markers are the creatine kinase-MB (CK-MB) fraction and the troponin levels Immediate treatment for suspected acute myocardial infarction includes oxygen, aspirin, and sublingual nitroglycerin.

Most cases of myocardial infarction with ST elevation on ECG (STEMI) are treated with reperfusion therapy, such as percutaneous coronary intervention (PCI) or thrombolysis. Non-ST elevation myocardial infarction (NSTEMI) may be managed with medication, although PCI may be required if the patient's risk warrants it. People who have multiple blockages of their coronary arteries, particularly if they also have diabetes mellitus, may benefit from bypass surgery (CABG). Treating the blockage causing the myocardial infarction by PCI and performing CABG later when the patient is more stable has been proposed. Rarely CABG may be preferred in the acute phase of myocardial infarction, for example when PCI has failed or is contraindicated.

Ischemic heart disease (which includes myocardial infarction, angina pectoris and heart failure when preceded by myocardial infarction) was the leading cause of death for both men and women worldwide in 2004 Important risk factors are previous cardiovascular disease, older age, tobacco smoking, high blood levels of certain lipids (low-density lipoprotein cholesterol, triglycerides) and low levels of high density lipoprotein (HDL) cholesterol, diabetes, high blood pressure, lack of physical activity and obesity, chronic kidney disease, excessive alcohol consumption, the abuse of illicit drugs (such as cocaine and amphetamines), and chronic high stress levels.

### Compounds of the Invention

Compounds, such as 4-sulfatase and arylsulfatase B are useful for the treatment of nervous system injury or traumas, such as spinal cord injury, stroke, or myocardial infarction. Without wishing to be bound by theory, these compounds may be particularly effective because they are capable of treating or preventing glial scar formation. These compounds are capable of increasing neuron or neurite growth and/or maintenance. In certain embodiments, a compound of the invention reduces the amount or activity of 4-sulfated GalNAc in a chondroitin glycosaminoglycan chain. One such therapeutic agent is 4-sulfatase which catalyzes the hydrolysis of sulfate from the C4 position of GalNAc and reduces 4S-GalNAc inhibitory activity on neuron or neurite growth and/or maintenance. Other therapeutic agents include antagonists of polypeptides involved in the biosynthesis of 4-sulfated chondroitin sulfate glycosaminoglycans (e.g., CHST11/12/13/14).

In certain embodiments, a compound of the invention can prevent, inhibit, or disrupt, or reduce by at least 10%, 25%, 50%, 75%, or 100% the inhibitory activity of 4S-GalNAc on neuron or neurite growth and/or maintenance.

In certain embodiments, a compound of the invention is a polypeptide. In other embodiments, a compound of the invention is a small molecule having a molecular weight less than about 1000 daltons, less than 800, less than 600, less than 500, less than 400, or less than about 300 daltons. Examples of compounds of the invention include 4-sulfatase and arylsulfatase B.

The term “pharmaceutically acceptable salt” also refers to a salt prepared from a compound of the invention having an acidic functional group, such as a carboxylic acid functional group, and a pharmaceutically acceptable inorganic or organic base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl,N-ethylamine; diethylamine; triethylamine; mono-, bis-, or tris-(2-hydroxy-lower alkyl amines), such as mono-, bis-, or tris-(2-hydroxyethyl)-amine, 2-hydroxy-tert-butylamine, or tris-(hydroxymethyl)methylamine, N, N,-di-lower alkyl-N-(hydroxy lower alkyl)-amines, such as N,N-dimethyl-N-(2-hydroxyethyl)-amine, or tri-(2-hydroxyethyl)amine; N-methyl-D-glucamine; and amino acids such as arginine, lysine, and the like. The term “pharmaceutically acceptable salt” also refers to a salt prepared from a compound disclosed herein, having a basic functional group, such as an amino functional group, and a pharmaceutically acceptable inorganic or organic acid. Suitable acids include, but are not limited to, hydrogen sulfate, citric acid, acetic acid, oxalic acid, hydrochloric acid, hydrogen bromide, hydrogen iodide, nitric acid, phosphoric acid, isonicotinic acid, lactic acid, salicylic acid, tartaric acid, ascorbic acid, succinic acid, maleic acid, besylic acid, fumaric acid, gluconic acid, glucaronic acid, saccharic acid, formic acid, benzoic acid, glutamic acid, methanesulfonic acid, ethanesulfonic acid, benzenesulfonic acid, and p-toluenesulfonic acid.

A composition for treatment of a central nervous system injury or trauma, such as a composition comprising arylsulfatase B, may be administered in combination with any other therapy for central nervous system injury or trauma therapy. If desired, agents of the invention are administered in combination with another therapy, conventional or otherwise, including but not limited to, surgery. Other therapies for central nervous system injury are being developed involving activated macrophages, stem cells, receptor blocking agents (e.g., antagonists, Riluzole, antibodies to NgR) and cAMP-PDE inhibitors. Conventional agents include, but are not limited to, growth factors or stem cell recruitment factors. Administration of progesterone is a potential therapy for treating traumatic brain injury, which is now in Phase III clinical trial.

### Polynucleotide Therapy

If desired, nucleic acid molecules that encode therapeutic polypeptides are delivered to nerve cells, glial cells, astrocytes or stem cells, such as endothelial stem cells, bone marrow-derived stem cells, hematopoietic stem cells, their precursors, or progenitors. In other approaches, nucleic acid molecules are delivered to cells of a tissue (e.g., Central Nervous System, spinal cord, brain, etc.). The nucleic acid molecules must be delivered to the cells of a subject in a form in which they can be taken up so that therapeutically effective levels of the therapeutic polypeptide (e.g., 4-sulfatase, arylsulfatase B) or fragment thereof can be produced.

A variety of expression systems exists for the production of therapeutic polypeptides. Expression vectors useful for producing such polypeptides include, without limitation, chromosomal, episomal, and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof.

One particular bacterial expression system for polypeptide production is the E. coli pET expression system (e.g., pET-28) (Novagen, Inc., Madison, Wis.). According to this expression system, DNA encoding a polypeptide is inserted into a pET vector in an orientation designed to allow expression. Since the gene encoding such a polypeptide is under the control of the T7 regulatory signals, expression of the polypeptide is achieved by inducing the expression of T7 RNA polymerase in the host cell. This is typically achieved using host strains that express T7 RNA polymerase in response to IPTG induction. Once produced, recombinant polypeptide is then isolated according to standard methods known in the art, for example, those described herein.

Another bacterial expression system for polypeptide production is the pGEX expression system (Pharmacia). This system employs a GST gene fusion system that is designed for high-level expression of genes or gene fragments as fusion proteins with rapid purification and recovery of functional gene products. The protein of interest is fused to the carboxyl terminus of the glutathione S-transferase protein from Schistosoma japonicum and is readily purified from bacterial lysates by affinity chromatography using Glutathione Sepharose 4B. Fusion proteins can be recovered under mild conditions by elution with glutathione. Cleavage of the glutathione 5-transferase domain from the fusion protein is facilitated by the presence of recognition sites for site-specific proteases upstream of this domain. For example, proteins expressed in pGEX-2T plasmids may be cleaved with thrombin; those expressed in pGEX-3X may be cleaved with factor Xa.

Alternatively, recombinant polypeptides of the invention are expressed in Pichia pastoris, a methylotrophic yeast. Pichia is capable of metabolizing methanol as the sole carbon source. The first step in the metabolism of methanol is the oxidation of methanol to formaldehyde by the enzyme, alcohol oxidase. Expression of this enzyme, which is coded for by the AOX1 gene is induced by methanol. The AOX1 promoter can be used for inducible polypeptide expression or the GAP promoter for constitutive expression of a gene of interest.

Once the recombinant polypeptide of the invention is expressed, it is isolated, for example, using affinity chromatography. In one example, an antibody (e.g., produced as described herein) raised against a polypeptide of the invention may be attached to a column and used to isolate the recombinant polypeptide. Lysis and fractionation of polypeptide-harboring cells prior to affinity chromatography may be performed by standard methods (see, e.g., Ausubel et al., supra). Alternatively, the polypeptide is isolated using a sequence tag, such as a hexahistidine tag, that binds to nickel column.

Once isolated, the recombinant protein can, if desired, be further purified, e.g., by high performance liquid chromatography (see, e.g., Fisher, Laboratory Techniques In Biochemistry and Molecular Biology, eds., Work and Burdon, Elsevier, 1980). Polypeptides of the invention, particularly short peptide fragments, can also be produced by chemical synthesis (e.g., by the methods described in Solid Phase Peptide Synthesis, 2nd ed., 1984 The Pierce Chemical Co., Rockford, Ill.). These general techniques of polypeptide expression and purification can also be used to produce and isolate useful peptide fragments or analogs (described herein).

If desired, a vector expressing stem cell recruiting factors is administered to a tissue or organ. SDF-1 (also called PBSF) (Campbell et al. (1998) Science 279(5349):381-4), 6-C-kine (also called Exodus-2), and MIP-3β (also called ELC or Exodus-3) induced adhesion of most circulating lymphocytes, including most CD4⁺ T cells; and MIP-3α (also called LARC or Exodus-1) triggered adhesion of memory, but not naive, CD4⁺ T cells. Tangemann et al. (1998) J. Immunol. 161:6330-7 disclose the role of secondary lymphoid-tissue chemokine (SLC), a high endothelial venule (HEV)-associated chemokine, with the homing of lymphocytes to secondary lymphoid organs. Campbell et al. (1998) J. Cell Biol. 141(4):1053-9 describe the receptor for SLC as CCR7, and that its ligand, SLC, can trigger rapid integrin-dependent arrest of lymphocytes rolling under physiological shear. In still other approaches, a vector encoding a polypeptide characteristically expressed in a cell of interest is introduced to a stem cell of the invention.

Transducing viral (e.g., retroviral, adenoviral, and adeno-associated viral) vectors can be used for somatic cell gene therapy, especially because of their high efficiency of infection and stable integration and expression (see, e.g., Cayouette et al., Human Gene Therapy 8:423-430, 1997; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, a polynucleotide encoding a stem cell recruiting factor, variant, or a fragment thereof, can be cloned into a retroviral vector and expression can be driven from its endogenous promoter, from the retroviral long terminal repeat, or from a promoter specific for a tissue or cell of interest. Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346). Most preferably, a viral vector is used to administer a therapeutic polynucleotide in pancreas, liver, heart, or another tissue or organ of interest.

Non-viral approaches can also be employed for the introduction of a therapeutic to a cell of a subject (e.g., a cell or tissue). For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Preferably the nucleic acids are administered in combination with a liposome and protamine.

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element. Exemplary constitutive promoters include the promoters for the following genes which encode certain constitutive or “housekeeping” functions: hypoxanthine phosphoribosyl transferase (HPRT), dihydrofolate reductase (DHFR) (Scharfmann et al., Proc. Natl. Acad. Sci. USA 88:4626-4630 (1991)), adenosine deaminase, phosphoglycerol kinase (PGK), pyruvate kinase, phosphoglycerol mutase, the actin promoter (Lai et al., Proc. Natl. Acad. Sci. USA 86: 10006-10010 (1989)), and other constitutive promoters known to those of skill in the art. In addition, many viral promoters function constitutively in eukaryotic cells. These include: the early and late promoters of SV40; the long terminal repeats (LTR) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

Genes that are under the control of inducible promoters are expressed only or to a greater degree, in the presence of an inducing agent, (e.g., transcription under control of the metallothionein promoter is greatly increased in presence of certain metal ions). Inducible promoters include responsive elements (REs) which stimulate transcription when their inducing factors are bound. For example, there are REs for serum factors, steroid hormones, retinoic acid and cyclic AMP. Promoters containing a particular RE can be chosen in order to obtain an inducible response and in some cases, the RE itself may be attached to a different promoter, thereby conferring inducibility to the recombinant gene. Thus, by selecting the appropriate promoter (constitutive versus inducible; strong versus weak), it is possible to control both the existence and level of expression of a therapeutic agent in the genetically modified stem cell and/or in a cell of the tissue having a deficiency in cell number. Selection and optimization of these factors for delivery of a therapeutically effective dose of a particular therapeutic agent is deemed to be within the scope of one of ordinary skill in the art without undue experimentation, taking into account the above-disclosed factors and the clinical profile of the subject.

In addition to at least one promoter and at least one heterologous nucleic acid encoding the therapeutic agent, the expression vector preferably includes a selection gene, for example, a neomycin resistance gene, for facilitating selection of stem cells that have been transfected or transduced with the expression vector.

If desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Another therapeutic approach included in the invention involves administration of a recombinant therapeutic, such as a recombinant stem cell recruiting factor, variant, or fragment thereof, either directly to the site of a potential or actual disease-affected tissue or systemically (for example, by any conventional recombinant protein administration technique). The dosage of the administered protein depends on a number of factors, including the size and health of the individual subject. For any particular subject, the specific dosage regimes should be adjusted over time according to the individual need and the professional judgment of the person administering or supervising the administration of the compositions.

### Inhibitory Nucleic Acids

Inhibitory nucleic acid molecules are those oligonucleotides that selectively inhibit the expression or activity of a polypeptide involved in chondroitin sulfate biosynthesis (e.g., N-acetyl galactosaminyl-4-O-sulfotransferases, CHST11/12/13/14). Such oligonucleotides include single and double stranded nucleic acid molecules (e.g., DNA, RNA, and analogs thereof) that are complementary to or that bind a nucleic acid molecule that encodes a polypeptide involved in chondroitin sulfate biosynthesis (e.g., N-acetyl galactosaminyl-4-O-sulfotransferases, CHST11/12/13/14) (e.g., antisense molecules, RNAi, siRNA, shRNA) as well as nucleic acid molecules that bind directly to a polypeptide involved in chondroitin sulfate biosynthesis (e.g., N-acetyl galactosaminyl-4-O-sulfotransferases, CHST11/12/13/14) to modulate its biological activity (e.g., aptamers).

siRNA

Short twenty-one to twenty-five nucleotide double-stranded RNAs are effective at down-regulating gene expression (Zamore et al., Cell 101: 25-33; Elbashir et al., Nature 411: 494-498, 2001, hereby incorporated by reference). The therapeutic effectiveness of an siRNA approach in mammals was demonstrated in vivo by McCaffrey et al. (Nature 418: 38-39.2002).

Given the sequence of a target gene, siRNAs may be designed to inactivate that gene. Such siRNAs, for example, could be administered directly to an affected tissue, or administered systemically. The nucleic acid sequence of a gene encoding an enzyme involved in chondroitin sulfate biosynthesis (e.g., N-acetyl galactosaminyl-4-O-sulfotransferases, CHST11/12/13/14) can be used to design small interfering RNAs (siRNAs). The 21 to 25 nucleotide siRNAs may be used, for example, as therapeutics to treat radiation exposure or a disorder thereof.

The inhibitory nucleic acid molecules of the present invention may be employed as double-stranded RNAs for RNA interference (RNAi)-mediated knock-down of expression of a gene or polypeptide encoding an enzyme involved in chondroitin sulfate biosynthesis (e.g., N-acetyl galactosaminyl-4-O-sulfotransferases, CHST11/12/13/14). RNAi is a method for decreasing the cellular expression of specific proteins of interest (reviewed in Tuschl, Chembiochem 2:239-245, 2001; Sharp, Genes & Devel. 15:485-490, 2000; Hutvagner and Zamore, Curr. Opin. Genet. Devel. 12:225-232, 2002; and Hannon, Nature 418:244-251, 2002). The introduction of siRNAs into cells either by transfection of dsRNAs or through expression of siRNAs using a plasmid-based expression system is increasingly being used to create loss-of-function phenotypes in mammalian cells.

### Ribozymes

Catalytic RNA molecules or ribozymes that include an antisense sequence of a gene encoding a polypeptide involved in chondroitin sulfate biosynthesis (e.g., N-acetyl galactosaminyl-4-O-sulfotransferases, CHST11/12/13/14) can be used to inhibit expression in vivo of a nucleic acid molecule encoding a polypeptide involved in chondroitin sulfate biosynthesis or the encoded polypeptide. The inclusion of ribozyme sequences within antisense RNAs confers RNA-cleaving activity upon them, thereby increasing the activity of the constructs. The design and use of target RNA-specific ribozymes is described in Haseloff et al., Nature 334:585-591. 1988, and U.S. Patent Application Publication No. 2003/0003469 A1, each of which is incorporated by reference.

Accordingly, the invention also features a catalytic RNA molecule that includes, in the binding arm, an antisense RNA having between eight and nineteen consecutive nucleobases. In preferred embodiments of this invention, the catalytic nucleic acid molecule is formed in a hammerhead or hairpin motif. Examples of such hammerhead motifs are described by Rossi et al., Aids Research and Human Retroviruses, 8:183, 1992. Example of hairpin motifs are described by Hampel et al., “RNA Catalyst for Cleaving Specific RNA Sequences,” filed Sep. 20, 1989, which is a continuation-in-part of U.S. Ser. No. 07/247,100 filed Sep. 20, 1988, Hampel and Tritz, Biochemistry, 28:4929, 1989, and Hampel et al., Nucleic Acids Research, 18: 299, 1990. These specific motifs are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule.

shRNA

Small hairpin RNAs consist of a stem-loop structure with optional 3′ UU-overhangs. While there may be variation, stems can range from 21 to 31 bp (desirably 25 to 29 bp), and the loops can range from 4 to 30 bp (desirably 4 to 23 bp). In one embodiment of the invention, the shRNA molecule is made that includes between eight and twenty-one consecutive nucleobases of a gene encoding an enzyme involved in chondroitin sulfate biosynthesis (e.g., N-acetyl galactosaminyl-4-O-sulfotransferases, CHST11/12/13/14).

For expression of shRNAs within cells, plasmid vectors containing either the polymerase III H1-RNA or U6 promoter, a cloning site for the stem-looped RNA insert, and a 4-5-thymidine transcription termination signal can be employed (e.g., pGeneClip Neomycin Vector; Promega Corporation). The Polymerase III promoters generally have well-defined initiation and stop sites and their transcripts lack poly(A) tails. The termination signal for these promoters is defined by the polythymidine tract, and the transcript is typically cleaved after the second uridine. Cleavage at this position generates a 3′ UU overhang in the expressed shRNA, which is similar to the 3′ overhangs of synthetic siRNAs.

For expression of shRNAs within cells, plasmid vectors containing either the polymerase III H1-RNA or U6 promoter, a cloning site for the stem-looped RNA insert, and a 4-5-thymidine transcription termination signal can be employed. The Polymerase III promoters generally have well-defined initiation and stop sites and their transcripts lack poly(A) tails. The termination signal for these promoters is defined by the polythymidine tract, and the transcript is typically cleaved after the second uridine. Cleavage at this position generates a 3′ UU overhang in the expressed shRNA, which is similar to the 3′ overhangs of synthetic siRNAs. Additional methods for expressing the shRNA in mammalian cells are described in the references cited above.

### Oligonucleotides and Other Nucleobase Oligomers

At least two types of oligonucleotides induce the cleavage of RNA by RNase H: polydeoxynucleotides with phosphodiester (PO) or phosphorothioate (PS) linkages. Although 2′-OMe-RNA sequences exhibit a high affinity for RNA targets, these sequences are not substrates for RNase H. A desirable oligonucleotide is one based on 2′-modified oligonucleotides containing oligodeoxynucleotide gaps with some or all internucleotide linkages modified to phosphorothioates for nuclease resistance. The presence of methylphosphonate modifications increases the affinity of the oligonucleotide for its target RNA and thus reduces the IC₅₀. This modification also increases the nuclease resistance of the modified oligonucleotide. It is understood that the methods and reagents of the present invention may be used in conjunction with any technologies that may be developed, including covalently-closed multiple antisense (CMAS) oligonucleotides (Moon et al., Biochem J. 346:295-303, 2000; PCT Publication No. WO 00/61595), ribbon-type antisense (RiAS) oligonucleotides (Moon et al., J. Biol. Chem. 275:4647-4653, 2000; PCT Publication No. WO 00/61595), and large circular antisense oligonucleotides (U.S. Patent Application Publication No. US 2002/0168631 A1).

As is known in the art, a nucleoside is a nucleobase-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn, the respective ends of this linear polymeric structure can be further joined to form a circular structure; open linear structures are generally preferred. Within the oligonucleotide structure, the phosphate groups are commonly referred to as forming the backbone of the oligonucleotide. The normal linkage or backbone of RNA and DNA is a 3′ to 5′ phosphodiester linkage.

Specific examples of preferred nucleobase oligomers useful in this invention include oligonucleotides containing modified backbones or non-natural internucleoside linkages. As defined in this specification, nucleobase oligomers having modified backbones include those that retain a phosphorus atom in the backbone and those that do not have a phosphorus atom in the backbone. For the purposes of this specification, modified oligonucleotides that do not have a phosphorus atom in their internucleoside backbone are also considered to be nucleobase oligomers.

Nucleobase oligomers that have modified oligonucleotide backbones include, for example, phosphorothioates, chiral phosphorothioates, phosphorodithioates, phosphotriesters, aminoalkyl-phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity, wherein the adjacent pairs of nucleoside units are linked 3′-5′ to 5′-3′ or 2′-5′ to 5′-2′. Various salts, mixed salts and free acid forms are also included. Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

Nucleobase oligomers having modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Representative United States patents that teach the preparation of the above oligonucleotides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

In other nucleobase oligomers, both the sugar and the internucleoside linkage, i.e., the backbone, are replaced with novel groups. The nucleobase units are maintained for hybridization with a nucleic acid molecule encoding a p75/TNF-α receptor or p55/TNF-α receptor. One such nucleobase oligomer, is referred to as a Peptide Nucleic Acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Methods for making and using these nucleobase oligomers are described, for example, in “Peptide Nucleic Acids: Protocols and Applications” Ed. P. E. Nielsen, Horizon Press, Norfolk, United Kingdom, 1999. Representative United States patents that teach the preparation of PNAs include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

In particular embodiments of the invention, the nucleobase oligomers have phosphorothioate backbones and nucleosides with heteroatom backbones, and in particular —CH₂—NH—O—CH₂—, —CH₂—N(CH₃)—O—CH₂— (known as a methylene (methylimino) or MMI backbone), —CH₂—O—N(CH₃)—CH₂—, —CH₂—N(CH₃)—N(CH₃)—CH₂—, and —O—N(CH₃)—CH₂—CH₂—. In other embodiments, the oligonucleotides have morpholino backbone structures described in U.S. Pat. No. 5,034,506.

Nucleobase oligomers may also contain one or more substituted sugar moieties. Nucleobase oligomers comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl, and alkynyl may be substituted or unsubstituted C₁ to C₁₀ alkyl or C₂ to C₁₀ alkenyl and alkynyl. Particularly preferred are O[(CH₂)ₙO]ₙCH₃, O(CH₂)ₙOCH₃, O(CH₂)ₙNH₂, O(CH₂)ₙCH₃, O(CH₂)ₙONH₂, and O(CH₂)nON[(CH₂)ₙCH₃)]₂, where n and m are from 1 to about 10. Other preferred nucleobase oligomers include one of the following at the 2′ position: C₁ to C₁₀ lower alkyl, substituted lower alkyl, alkaryl, aralkyl, O-alkaryl, or O-aralkyl, SH, SCH₃, OCN, Cl, Br, CN, CF₃, OCF₃, SOCH₃, SO₂CH₃, ONO₂, NO₂, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of a nucleobase oligomer, or a group for improving the pharmacodynamic properties of an nucleobase oligomer, and other substituents having similar properties. Preferred modifications are 2′-O-methyl and 2′-methoxyethoxy (2′-O—CH₂CH₂OCH₃, also known as 2′-O-(2-methoxyethyl) or 2′-MOE). Another desirable modification is 2′-dimethylaminooxyethoxy (i.e., O(CH₂)₂ON(CH₃)₂), also known as 2′-DMAOE. Other modifications include, 2′-aminopropoxy (2′-OCH₂CH₂CH₂NH₂) and 2′-fluoro (2′-F). Similar modifications may also be made at other positions on an oligonucleotide or other nucleobase oligomer, particularly the 3′ position of the sugar on the 3′ terminal nucleotide or in 2′-5′ linked oligonucleotides and the 5′ position of 5′ terminal nucleotide. Nucleobase oligomers may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

Nucleobase oligomers may also include nucleobase modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases, such as 5-methylcytosine (5-me-C), 5-hydroxymethyl cytosine, xanthine, hypoxanthine, 2-aminoadenine, 6-methyl and other alkyl derivatives of adenine and guanine; 2-propyl and other alkyl derivatives of adenine and guanine; 2-thiouracil, 2-thiothymine and 2-thiocytosine; 5-halouracil and cytosine; 5-propynyl uracil and cytosine; 6-azo uracil, cytosine and thymine; 5-uracil (pseudouracil); 4-thiouracil; 8-halo, 8-amino, 8-thiol, 8-thioalkyl, 8-hydroxyl and other 8-substituted adenines and guanines; 5-halo (e.g., 5-bromo), 5-trifluoromethyl and other 5-substituted uracils and cytosines; 7-methylguanine and 7-methyladenine; 8-azaguanine and 8-azaadenine; 7-deazaguanine and 7-deazaadenine; and 3-deazaguanine and 3-deazaadenine. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of an antisense oligonucleotide of the invention. These include 5-substituted pyrimidines, 6-azapyrimidines, and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278) and are desirable base substitutions, even more particularly when combined with 2′-O-methoxyethyl or 2′-O-methyl sugar modifications. Representative United States patents that teach the preparation of certain of the above noted modified nucleobases as well as other modified nucleobases include U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; and 5,750,692, each of which is herein incorporated by reference.

Another modification of a nucleobase oligomer of the invention involves chemically linking to the nucleobase oligomer one or more moieties or conjugates that enhance the activity, cellular distribution, or cellular uptake of the oligonucleotide. Such moieties include but are not limited to lipid moieties such as a cholesterol moiety (Letsinger et al., Proc. Natl. Acad. Sci. USA, 86:6553-6556, 1989), cholic acid (Manoharan et al., Bioorg. Med. Chem. Let, 4:1053-1060, 1994), a thioether, e.g., hexyl-S-tritylthiol (Manoharan et al., Ann. N.Y. Acad. Sci., 660:306-309, 1992; Manoharan et al., Bioorg. Med. Chem. Let., 3:2765-2770, 1993), a thiocholesterol (Oberhauser et al., Nucl. Acids Res., 20:533-538: 1992), an aliphatic chain, e.g., dodecandiol or undecyl residues (Saison-Behmoaras et al., EMBO J., 10:1111-1118, 1991; Kabanov et al., FEBS Lett., 259:327-330, 1990; Svinarchuk et al., Biochimie, 75:49-54, 1993), a phospholipid, e.g., di-hexadecyl-rac-glycerol or triethylammonium 1,2-di-O-hexadecyl-rac-glycero-3-H-phosphonate (Manoharan et al., Tetrahedron Lett., 36:3651-3654, 1995; Shea et al., Nucl. Acids Res., 18:3777-3783, 1990), a polyamine or a polyethylene glycol chain (Manoharan et al., Nucleosides & Nucleotides, 14:969-973, 1995), or adamantane acetic acid (Manoharan et al., Tetrahedron Lett., 36:3651-3654, 1995), a palmityl moiety (Mishra et al., Biochim Biophys. Acta, 1264:229-237, 1995), or an octadecylamine or hexylamino-carbonyl-oxycholesterol moiety (Crooke et al., J. Pharmacol. Exp. Ther., 277:923-937, 1996. Representative United States patents that teach the preparation of such nucleobase oligomer conjugates include U.S. Pat. Nos. 4,587,044; 4,605,735; 4,667,025; 4,762,779; 4,789,737; 4,824,941; 4,828,979; 4,835,263; 4,876,335; 4,904,582; 4,948,882; 4,958,013; 5,082,830; 5,109,124; 5,112,963; 5,118,802; 5,138,045; 5,214,136; 5,218,105; 5,245,022; 5,254,469; 5,258,506; 5,262,536; 5,272,250; 5,292,873; 5,317,098; 5,371,241, 5,391,723; 5,414,077; 5,416,203, 5,451,463; 5,486,603; 5,510,475; 5,512,439; 5,512,667; 5,514,785; 5,525,465; 5,541,313; 5,545,730; 5,552,538; 5,565,552; 5,567,810; 5,574,142; 5,578,717; 5,578,718; 5,580,731; 5,585,481; 5,587,371; 5,591,584; 5,595,726; 5,597,696; 5,599,923; 5,599,928; 5,608,046; and 5,688,941, each of which is herein incorporated by reference.

The present invention also includes nucleobase oligomers that are chimeric compounds. “Chimeric” nucleobase oligomers are nucleobase oligomers, particularly oligonucleotides, that contain two or more chemically distinct regions, each made up of at least one monomer unit, i.e., a nucleotide in the case of an oligonucleotide. These nucleobase oligomers typically contain at least one region where the nucleobase oligomer is modified to confer, upon the nucleobase oligomer, increased resistance to nuclease degradation, increased cellular uptake, and/or increased binding affinity for the target nucleic acid. An additional region of the nucleobase oligomer may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of nucleobase oligomer inhibition of gene expression. Consequently, comparable results can often be obtained with shorter nucleobase oligomers when chimeric nucleobase oligomers are used, compared to phosphorothioate deoxyoligonucleotides hybridizing to the same target region.

Chimeric nucleobase oligomers of the invention may be formed as composite structures of two or more nucleobase oligomers as described above. Such nucleobase oligomers, when oligonucleotides, have also been referred to in the art as hybrids or gapmers. Representative United States patents that teach the preparation of such hybrid structures include U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference in its entirety.

The nucleobase oligomers used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

The nucleobase oligomers of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative United States patents that teach the preparation of such uptake, distribution and/or absorption assisting formulations include U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

### Delivery of Nucleobase Oligomers

Naked inhibitory nucleic acid molecules, or analogs thereof, are capable of entering mammalian cells and inhibiting expression of a gene of interest. Nonetheless, it may be desirable to utilize a formulation that aids in the delivery of oligonucleotides or other nucleobase oligomers to cells (see, e.g., U.S. Pat. Nos. 5,656,611, 5,753,613, 5,785,992, 6,120,798, 6,221,959, 6,346,613, and 6,353,055, each of which is hereby incorporated by reference).

### Non-Polypeptide and Small Molecule Agents

The invention also provides small organic molecules, such as those obtained from natural products, or those compounds synthesized by conventional organic synthesis or combinatorial organic synthesis that selectively bind to a polypeptide involved in chondroitin sulfate biosynthesis (e.g., N-acetyl galactosaminyl-4-O-sulfotransferases, CHST11/12/13/14) or 4-sulfate modification (e.g., a 4-sulfated N-acetylgalactosyl residue) and thereby inhibit the inhibitory activity of 4-sulfated CS GAGs on nerve growth and/or proliferation. Compounds can be tested for their ability to bind to a polypeptide involved in chondroitin sulfate biosynthesis or 4-sulfate for example, by using a column binding technique. Such screening methods are well known in the art, and are described herein. Targeting such agents can provide small molecules or other compounds that inhibit the repellent activity of 4-sulfated glycosaminoglycan chains on neurons.

Other suitable non-peptidic compounds include, for example, oligonucleotides. Oligonucleotides as used herein refers to any heteropolymeric material containing purine, pyrimidine and other aromatic bases. DNA and RNA oligonucleotides are suitable for use with the invention, as are oligonucleotides with sugar (e.g., 2′ alkylated riboses) and backbone modifications (e.g., phosphorothioate oligonucleotides). Oligonucleotides may present commonly found purine and pyrimidine bases such as adenine, thymine, guanine, cytidine and uridine, as well as bases modified within the heterocyclic ring portion (e.g., 7-deazaguanine) or in exocyclic positions. Oligonucleotide also encompasses heteropolymers with distinct structures that also present aromatic bases, including polyamide nucleic acids and the like.

An oligonucleotide antagonist of the invention can be generated by a number of methods known to one of skill in the art. In one embodiment, a pool of oligonucleotides is generated containing a large number of sequences. Pools can be generated, for example, by solid phase synthesis using mixtures of monomers at an elongation step. The pool of oligonucleotides is sorted by passing a solution-containing the pool over a solid matrix to which a target molecule (e.g., 4-sulfated N-acetylgalactosyl or polypeptide involved in chondroitin sulfate biosynthesis or fragment thereof has been affixed). Sequences within the pool that bind to the target molecule are retained on the solid matrix. These sequences are eluted with a solution of different salt concentration or pH. The column retains those sequences that bind the target molecule, thus, enriching the pool for sequences specific for the target molecule. Additional selection steps may be used to further enrich for sequences having desired activity. The pool can be amplified and, if necessary, mutagenized and the process repeated until the pool shows the characteristics of an antagonist of the invention. Individual antagonists can be identified by sequencing members of the oligonucleotide pool, usually after cloning said sequences into a host organism such as E. coli.

### Antibodies

Antibodies that selectively bind 4-sulfated GalNAc or a peptide involved in chondroitin sulfate biosynthesis (e.g., N-acetyl galactosaminyl-4-O-sulfotransferases, CHST11/12/13/14) and inhibit its activity are useful in the methods of the invention. In one embodiment, selective binding of antibody to a peptide involved in chondroitin sulfate biosynthesis reduces biosynthesis of 4-sulfated CS GAGs. In another embodiment, selective binding of antibody to 4-sulfate modification inhibits its repellent activity, e.g., as assayed by a nerve growth or proliferation assay.

Methods of preparing antibodies are well known to those of ordinary skill in the science of immunology. As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fc fragment of intact antibody, clear more rapidly from the circulation, and may have less non-specific tissue binding of an intact antibody (Wahl et al., J. Nucl. Med. 24:316-325 (1983). The antibodies of the invention comprise whole native antibodies, bispecific antibodies; chimeric antibodies; Fab, Fab′, single chain V region fragments (scFv), fusion polypeptides, and unconventional antibodies.

In one embodiment, the antibody is a polyclonal antibody. The preparation and use of polyclonal antibodies are known to the skilled artisan. The invention also encompasses hybrid antibodies, in which one pair of heavy and light chains is obtained from a first antibody, while the other pair of heavy and light chains is obtained from a different second antibody. Such hybrids may also be formed using humanized heavy and light chains. Such antibodies are often referred to as “chimeric” antibodies.

In general, intact antibodies are said to contain “Fc” and “Fab” regions. The Fc regions are involved in complement activation and are not involved in antigen binding. An antibody from which the Fc′ region has been enzymatically cleaved, or which has been produced without the Fc′ region, designated an “F(ab′)₂” fragment, retains both of the antigen binding sites of the intact antibody. Similarly, an antibody from which the Fc region has been enzymatically cleaved, or which has been produced without the Fc region, designated an “Fab′” fragment, retains one of the antigen binding sites of the intact antibody. Fab′ fragments consist of a covalently bound antibody light chain and a portion of the antibody heavy chain, denoted “Fd.” The Fd fragments are the major determinants of antibody specificity (a single Fd fragment may be associated with up to ten different light chains without altering antibody specificity). Isolated Fd fragments retain the ability to specifically bind to immunogenic epitopes.

Antibodies can be made by any of the methods known in the art utilizing a polypeptide involved in chondroitin sulfate biosynthesis (e.g., N-acetyl galactosaminyl-4-O-sulfotransferases, CHST11/12/13/14), or fragments thereof. One method of obtaining antibodies is to immunize suitable host animals with an immunogen and to follow standard procedures for polyclonal or monoclonal antibody production. The immunogen will facilitate presentation of the immunogen on the cell surface. Immunization of a suitable host can be carried out in a number of ways. Nucleic acid sequences encoding a polypeptide involved in chondroitin sulfate biosynthesis or immunogenic fragments thereof, can be provided to the host in a delivery vehicle that is taken up by immune cells of the host. The cells will in turn express the receptor on the cell surface generating an immunogenic response in the host. Alternatively, nucleic acid sequences encoding a polypeptide involved in chondroitin sulfate biosynthesis (e.g., N-acetyl galactosaminyl-4-O-sulfotransferases, CHST11/12/13/14), or immunogenic fragments thereof, can be expressed in cells in vitro, followed by isolation of the polypeptide and administration of the polypeptide to a suitable host in which antibodies are raised.

Alternatively, antibodies may, if desired, be derived from an antibody phage display library. A bacteriophage is capable of infecting and reproducing within bacteria, which can be engineered, when combined with human antibody genes, to display human antibody proteins. Phage display is the process by which the phage is made to ‘display’ the human antibody proteins on its surface. Genes from the human antibody gene libraries are inserted into a population of phage. Each phage carries the genes for a different antibody and thus displays a different antibody on its surface.

Antibodies made by any method known in the art can then be purified from the host. Antibody purification methods may include salt precipitation (for example, with ammonium sulfate), ion exchange chromatography (for example, on a cationic or anionic exchange column preferably run at neutral pH and eluted with step gradients of increasing ionic strength), gel filtration chromatography (including gel filtration HPLC), and chromatography on affinity resins such as protein A, protein G, hydroxyapatite, and anti-immunoglobulin.

Antibodies can be conveniently produced from hybridoma cells engineered to express the antibody. Methods of making hybridomas are well known in the art. The hybridoma cells can be cultured in a suitable medium, and spent medium can be used as an antibody source. Polynucleotides encoding the antibody of interest can in turn be obtained from the hybridoma that produces the antibody, and then the antibody may be produced synthetically or recombinantly from these DNA sequences. For the production of large amounts of antibody, it is generally more convenient to obtain an ascites fluid. The method of raising ascites generally comprises injecting hybridoma cells into an immunologically naive histocompatible or immunotolerant mammal, especially a mouse. The mammal may be primed for ascites production by prior administration of a suitable composition (e.g., Pristane).

Monoclonal antibodies (Mabs) produced by methods of the invention can be “humanized” by methods known in the art. “Humanized” antibodies are antibodies in which at least part of the sequence has been altered from its initial form to render it more like human immunoglobulins. Techniques to humanize antibodies are particularly useful when non-human animal (e.g., murine) antibodies are generated. Examples of methods for humanizing a murine antibody are provided in U.S. Pat. Nos. 4,816,567, 5,530,101, 5,225,539, 5,585,089, 5,693,762 and 5,859,205.

In other embodiments, the invention provides “unconventional antibodies.” Unconventional antibodies include, but are not limited to, nanobodies, linear antibodies (Zapata et al., Protein Eng. 8(10): 1057-1062,1995), single domain antibodies, single chain antibodies, and antibodies having multiple valencies (e.g., diabodies, tribodies, tetrabodies, and pentabodies). Nanobodies are the smallest fragments of naturally occurring heavy-chain antibodies that have evolved to be fully functional in the absence of a light chain. Nanobodies have the affinity and specificity of conventional antibodies although they are only half of the size of a single chain Fv fragment. The consequence of this unique structure, combined with their extreme stability and a high degree of homology with human antibody frameworks, is that nanobodies can bind therapeutic targets not accessible to conventional antibodies. Recombinant antibody fragments with multiple valencies provide high binding avidity and unique targeting specificity to cancer cells. These multimeric scFvs (e.g., diabodies, tetrabodies) offer an improvement over the parent antibody since small molecules of ˜60-100 kDa in size provide faster blood clearance and rapid tissue uptake See Power et al., (Generation of recombinant multimeric antibody fragments for tumor diagnosis and therapy. Methods Mol Biol, 207, 335-50, 2003); and Wu et al. (Anti-carcinoembryonic antigen (CEA) diabody for rapid tumor targeting and imaging. Tumor Targeting, 4, 47-58, 1999).

Various techniques for making unconventional antibodies have been described. Bispecific antibodies produced using leucine zippers are described by Kostelny et al. (J. Immunol. 148(5):1547-1553, 1992). Diabody technology is described by Hollinger et al. (Proc. Natl. Acad. Sci. USA 90:6444-6448, 1993). Another strategy for making bispecific antibody fragments by the use of single-chain Fv (sFv) diners is described by Gruber et al. (J. Immunol. 152:5368, 1994). Trispecific antibodies are described by Tutt et al. (J. Immunol. 147:60, 1991). Single chain Fv polypeptide antibodies include a covalently linked VH::VL heterodimer which can be expressed from a nucleic acid including V_{H}- and V_{L}-encoding sequences either joined directly or joined by a peptide-encoding linker as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754.

### Test Compounds and Extracts

The invention further provides a simple means for identifying agents (including nucleic acids, peptides, small molecule inhibitors, and mimetics) that are capable of decreasing the activity of a 4-sulfate modified glycosaminoglyan on neuron or neurite growth and/or maintenance, for example, catalyzing the hydrolysis of 4-sulfate in a 4-sulfated GalNAc of an glycosaminoglyan or binding to a 4-sulfate modified GalNAc in a sulfated glycosaminoglyan. Such compounds are also expected to be useful for the treatment or prevention of a central nervous system injury or trauma (e.g., spinal cord injury).

In general, antagonists of 4-sulfate modified chondroitin sulfate (e.g., agents that specifically bind and/or reduce the activity of 4-sulfate modified chondroitin sulfate) or its biosynthesis and other agents that enhance the efficacy of an agent described herein may be identified from large libraries of natural product or synthetic (or semi-synthetic) extracts or chemical libraries or from polypeptide or nucleic acid libraries, according to methods known in the art. Those skilled in the field of drug discovery and development will understand that the precise source of test extracts or compounds is not critical to the screening procedure(s) of the invention. Agents used in screens may include those known as therapeutics for neuron or neurite growth and/or migration. Alternatively, virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as the modification of existing polypeptides.

Libraries of natural polypeptides in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). Such polypeptides can be modified to include a protein transduction domain using methods known in the art and described herein. In addition, natural and synthetically produced libraries are produced, if desired, according to methods known in the art, e.g., by standard extraction and fractionation methods. Examples of methods for the synthesis of molecular libraries can be found in the art, for example in: DeWitt et al., Proc. Natl. Acad. Sci. U.S.A. 90:6909, 1993; Erb et al., Proc. Natl. Acad. Sci. USA 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37:1233, 1994. Furthermore, if desired, any library or compound is readily modified using standard chemical, physical, or biochemical methods.

Numerous methods are also available for generating random or directed synthesis (e.g., semi-synthesis or total synthesis) of any number of polypeptides, chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, chemical compounds to be used as candidate compounds can be synthesized from readily available starting materials using standard synthetic techniques and methodologies known to those of ordinary skill in the art. Synthetic chemistry transformations and protecting group methodologies (protection and deprotection) useful in synthesizing the compounds identified by the methods described herein are known in the art and include, for example, those such as described in R. Larock, Comprehensive Organic Transformations, VCH Publishers (1989); T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Libraries of compounds may be presented in solution (e.g., Houghten, Biotechniques 13:412-421, 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc Natl Acad Sci USA 89:1865-1869, 1992) or on phage (Scott and Smith, Science 249:386-390, 1990; Devlin, Science 249:404-406, 1990; Cwirla et al. Proc. Natl. Acad. Sci. 87:6378-6382, 1990; Felici, J. Mol. Biol. 222:301-310, 1991; Ladner supra.).

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity should be employed whenever possible.

When a crude extract is found to have antagonistic or binding activity of 4-sulfate modified chondroitin sulfate, further fractionation of the positive lead extract is necessary to isolate molecular constituents responsible for the observed effect. Thus, the goal of the extraction, fractionation, and purification process is the careful characterization and identification of a chemical entity within the crude extract that promotes nerve cell proliferation or viability in the presence of 4-sulfated CS GAGs. Methods of fractionation and purification of such heterogenous extracts are known in the art. If desired, compounds shown to be useful as therapeutics are chemically modified according to methods known in the art.

### Pharmaceutical Therapeutics

In other embodiments, agents discovered to have medicinal value using the methods described herein are useful as a drug or as information for structural modification of existing compounds, e.g., by rational drug design. Such methods are useful for screening agents having an effect on neuron or neurite growth and/or maintenance.

For therapeutic uses, the compositions or agents identified using the methods disclosed herein may be administered locally (e.g., at the site of injury) or systemically, for example, formulated in a pharmaceutically-acceptable buffer such as physiological saline. Preferable routes of administration include, for example, subcutaneous, intravenous, interperitoneally, intramuscular, or intradermal injections that provide continuous, sustained levels of the drug in the patient. Treatment of human patients or other animals will be carried out using a therapeutically effective amount of a therapeutic identified herein in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of nervous system injury or trauma. Generally, amounts will be in the range of those used for other agents used in the treatment of other conditions associated with nervous system injury or trauma, although in certain instances lower amounts will be needed because of the increased specificity of the compound. A compound is administered at a dosage that reduces glial scarring and/or increases neuron or neurite growth and/or migration.

### Formulation of Pharmaceutical Compositions

The administration of a compound or a combination of compounds for the neuronal growth may be by any suitable means that results in a concentration of the therapeutic that, combined with other components, is effective in treatment of nervous system injury. The compound may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Human dosage amounts can initially be determined by extrapolating from the amount of compound used in, for example, mice, as a skilled artisan recognizes it is routine in the art to modify the dosage for humans compared to animal models. In certain embodiments it is envisioned that the dosage may vary from between about 1 μg compound/Kg body weight to about 5000 mg compound/Kg body weight; or from about 5 mg/Kg body weight to about 4000 mg/Kg body weight or from about 10 mg/Kg body weight to about 3000 mg/Kg body weight; or from about 50 mg/Kg body weight to about 2000 mg/Kg body weight; or from about 100 mg/Kg body weight to about 1000 mg/Kg body weight; or from about 150 mg/Kg body weight to about 500 mg/Kg body weight. In other embodiments this dose may be about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, or 5000 mg/Kg body weight. In other embodiments, it is envisaged that doses may be in the range of about 5 mg compound/Kg body to about 20 mg compound/Kg body. In other embodiments the doses may be about 8, 10, 12, 14, 16 or 18 mg/Kg body weight. Of course, the therapeutically effective amount may be optimized via upward or downward adjustments, as is routinely done in such treatment protocols, depending upon the specific condition and severity thereof.

The effective amount of a therapeutic agent (e.g., 4-sulfatase) can be administered in a single dosage, two dosages or a plurality of dosages. Although it is to be understood that the dosage may be administered at any time, in one embodiment, the dosage is administered within 0.5, 1, 2, 3, 4, 5, 6, 7, 8, 9, 10, 11, or 12 hours after injury, or as soon as is feasible. In another embodiment, the dosage is administered to an injured mammal in one, two or a plurality of dosages; such dosages would be dependent on the severity of the injury and the amount of CSPGs present in the glial scarring. Where a plurality of dosages is administered, they may be delivered on a daily, weekly, or bi-weekly basis. The delivery of the dosages may be by means of catheter or syringe. Alternatively, the treatment can be administered during surgery to allow direct application to the glial scar.

Pharmaceutical compositions according to the invention may be formulated to release the active compound substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with the thymus; (v) formulations that allow for convenient dosing, such that doses are administered, for example, once every one or two weeks; and (vi) formulations that target central nervous system injury or trauma by using carriers or chemical derivatives to deliver the therapeutic agent to a particular cell type (e.g., neuron). For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

The compounds of the present invention can also be administered in combination with other active ingredients, such as, for example, adjuvants, protease inhibitors, or other compatible drugs or compounds where such combination is seen to be desirable or advantageous in achieving the desired effects of the methods described herein.

### Combination Therapies

In some cases, the agents and pharmaceutical compositions described herein are administered in combination with another therapeutic molecule. The therapeutic molecule can be any compound used to treat CNS injury or secondary complications as a result thereof. Examples of these compounds include those used for the treatment of spinal cord injury, such as steroids, e.g., methylprednisolone (Medrol®). Examles of compounds used for the treatment of brain injury include diuretics, anti-seizure drugs, and coma-inducing drugs. Other compounds used for the treatment of brain injury include those that treat ischemia (insufficient blood flow), cerebral hypoxia (insufficient oxygen in the brain), hypotension (low blood pressure), cerebral edema (swelling of the brain), changes in the blood flow to the brain, and raised intracranial pressure (the pressure within the skull). Examples of compounds used for the treatment of stroke include anti-clotting agents and anti-platelet drugs. Compounds utilized to treat secondary complications as a result of CNS injury also include those used to treat bladder incontinence, bowel incontinence, bed sores, etc. The phrase “combination” also embraces groups of compounds or non-drug therapies useful as part of a combination therapy.

The phrase “combination therapy” embraces the administration of an agent that reduces the amount or activity of a 4-sulfated GalNAc in a chondroitin glycosaminoglycan chain (e.g., ARSB) and a second therapeutic agent as part of a specific treatment regimen intended to provide a beneficial effect from the co-action of these therapeutic agents. The beneficial effect of the combination includes, but is not limited to, pharmacokinetic or pharmacodynamic co-action resulting from the combination of therapeutic agents.

Administration of these therapeutic agents in combination typically is carried out over a defined time period (usually minutes, hours, days, or weeks depending upon the combination selected). In some cases, the agent that reduces the amount or activity of a 4-sulfated GalNAc in a chondroitin glycosaminoglycan chain is administered before, during, or after administration of the additional therapeutic agent. For example, ARSB is administered before the first administration of the additional therapeutic agent (e.g., 1, 2, 3, 4, 5, 6, 7, 8, 9, 10, 11, 12, 13, or 14 days or more before). Alternatively, ARSB is administered after the first administration of the additional therapeutic agent (e.g., 1, 2, 3, 4, 5, 6, 7, 8, 9, 10, 11, 12, 13, or 14 days or more after). In some cases, ARSB is administered simultaneously with the first administration of the additional therapeutic agent.

“Combination therapy” generally is not intended to encompass the administration of two or more of these therapeutic agents as part of separate monotherapy regimens that incidentally and arbitrarily result in the combinations of the present invention. “Combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a fixed ratio of each therapeutic agent in combination or in multiple, single doses for each of the therapeutic agents. For example, one combination of the present invention comprises an agent that reduces the amount or activity of a 4-sulfated GalNAc in a chondroitin glycosaminoglycan chain (e.g., ARSB) and at least one additional therapeutic agent (e.g., an anti-seizure drug, an anti-clotting drug, an anti-platelet drug, or a bladder incontinence drug) at the same or different times or they can be formulated as a single, co-formulated pharmaceutical composition comprising the two compounds. As another example, a combination of the present invention (e.g., an agent that reduces the amount or activity of a 4-sulfated GalNAc in a chondroitin glycosaminoglycan chain and at least one additional therapeutic agent) is formulated as separate pharmaceutical compositions that can be administered at the same or different time. Sequential or substantially simultaneous administration of each therapeutic agent can be effected by any appropriate route including, but not limited to, oral routes, intravenous routes, intramuscular routes, direct absorption through mucous membrane tissues (e.g., nasal, mouth, vaginal, and rectal), and ocular routes (e.g., intravitreal, intraocular, etc.). The therapeutic agents can be administered by the same route or by different routes. For example, one component of a particular combination may be administered by intravenous injection while the other component(s) of the combination may be administered directly into the spinal cord. The components may be administered in any therapeutically effective sequence.

The amount of therapeutic agent administered to a subject can readily be determined by the attending physician or veterinarian. Generally, an efficacious or effective amount of an agent that reduces the amount or activity of a 4-sulfated GalNAc in a chondroitin glycosaminoglycan chain and an additional therapeutic is determined by first administering a low dose of one or both active agents and then incrementally increasing the administered dose or dosages until a desired effect is observed (e.g., reduced symptoms associated with CNS injury), with minimal or no toxic side effects. Applicable methods for determining an appropriate dose and dosing schedule for administration of a combination of the present invention are described, for example, in Goodman and Gilman's The Pharmacological Basis of Therapeutics, 11th Edition., supra, and in Remington: The Science and Practice of Pharmacy, 20th and 21st Editions, supra.

The recitation of an embodiment for a variable or aspect herein includes that embodiment as any single embodiment or in combination with any other embodiments or portions thereof.

### Parenteral Compositions

The pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. Preferably, the composition may be administered locally, at or near the site of injury. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent that reduces or ameliorates a nervous system injury or trauma, the composition may include suitable parenterally acceptable carriers and/or excipients. The active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

As indicated above, the pharmaceutical compositions according to the invention may be in the form suitable for sterile injection. To prepare such a composition, the suitable active therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

### Controlled Release Parenteral Compositions

Controlled release parenteral compositions may be in form of aqueous suspensions, microspheres, microcapsules, magnetic microspheres, oil solutions, oil suspensions, or emulsions. Alternatively, the active drug may be incorporated in biocompatible carriers, liposomes, nanoparticles, implants, or infusion devices.

Materials for use in the preparation of microspheres and/or microcapsules are, e.g., biodegradable/bioerodible polymers such as polygalactin, poly-(isobutyl cyanoacrylate), poly(2-hydroxyethyl-L-glutam-nine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

The present invention provides methods of treating central nervous system injury, disease and/or disorders or symptoms thereof which comprise administering a therapeutically effective amount of a pharmaceutical composition comprising a compound of the formulae herein to a subject (e.g., a mammal such as a human). Thus, one embodiment is a method of treating a subject suffering from or susceptible to a central nervous system injury, disease or disorder or symptom thereof. The method includes the step of administering to the mammal a therapeutic amount of a compound herein sufficient to treat the disease or disorder or symptom thereof, under conditions such that the disease or disorder is treated.

The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention (which include prophylactic treatment) in general comprise administration of a therapeutically effective amount of the compounds herein, such as a compound of the formulae herein to a subject (e.g., animal, human) in need thereof, including a mammal, particularly a human. Such treatment will be suitably administered to subjects, particularly humans, suffering from, having, susceptible to, or at risk for a central nervous system injury, disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The compounds herein may be also used in the treatment of any other disorders in which stroke or myocardial infarction may be implicated.

In one embodiment, the invention provides a method of monitoring treatment progress. The method includes the step of determining a level of diagnostic marker (Marker) (e.g., any target delineated herein modulated by a compound herein, a protein or indicator thereof, etc.) or diagnostic measurement (e.g., screen, assay) in a subject suffering from or susceptible to a disorder or symptoms thereof associated with stroke or myocardial infarction in which the subject has been administered a therapeutic amount of a compound herein sufficient to treat the condition or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

### Solid Dosage Forms For Oral Use

Formulations for oral use include tablets containing the active ingredient(s) (e.g., small molecule inhibitors of 4-sulfation; chlorate) in a mixture with non-toxic pharmaceutically acceptable excipients. Such formulations are known to the skilled artisan. Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active drug in a predetermined pattern (e.g., in order to achieve a controlled release formulation) or it may be adapted not to release the active drug until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material, such as, e.g., glyceryl monostearate or glyceryl distearate may be employed.

The solid tablet compositions may include a coating adapted to protect the composition from unwanted chemical changes, (e.g., chemical degradation prior to the release of the active therapeutic substance). The coating may be applied on the solid dosage form in a similar manner as that described in Encyclopedia of Pharmaceutical Technology, supra.

Formulations for oral use may also be presented as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus or a spray drying equipment.

### Controlled Release Oral Dosage Forms

Controlled release compositions for oral use may, e.g., be constructed to release the active therapeutic by controlling the dissolution and/or the diffusion of the active substance. Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated metylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

A controlled release composition containing one or more therapeutic compounds may also be in the form of a buoyant tablet or capsule (i.e., a tablet or capsule that, upon oral administration, floats on top of the gastric content for a certain period of time). A buoyant tablet formulation of the compound(s) can be prepared by granulating a mixture of the compound(s) with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

### Methods of Treatment

In one embodiment, the present invention provides a method of treating a Central Nervous System injury or trauma (e.g., spinal cord injury). Advantageously, the invention provides methods for increasing neuron or neurite growth and/or migration, which may be used for treating neuronal injury or trauma. Another aspect of the invention is the use of a compound of the invention in the manufacture of a medicament for increasing neuronal or neurite growth and/or migration in a subject. For example, in addition to treating Central Nervous System injury or trauma, the present invention may also be used in the treatment of stroke or myocardial infarction, conditions which have the potential to cause neuronal injury or trauma. The methods involve administering to a subject having a neuron injury an effective amount of a therapeutic agent of the invention, for example, a composition comprising human arylsulfatase B. Preferably, such agents are administered as part of a composition additionally comprising a pharmaceutically acceptable carrier. Therapeutic agents may be administered locally at the site of injury (e.g., nucleic acids via liposomal or viral delivery, polypeptides, antibodies) or systemically (e.g., small molecule inhibitors, antibodies) as to be effective, as is known to those skilled in the art. Preferably this method is employed to treat a subject suffering from or susceptible to neuron injury or trauma. Furthermore, the treatment methods of the invention can be used in combination with other available therapies for treating central nervous system injuries. Other embodiments include any of the methods herein wherein the subject is identified as in need of the indicated treatment. After a subject is diagnosed as having a central nervous system injury (e.g., a spinal cord injury) or other type of condition causing neuron injury (e.g., stroke or myocardial infarction) a method of treatment is selected. Preferably, the medicament is used for treatment or prevention in a subject of a disease, disorder or symptom set forth above. Thus, the invention provides methods for selecting a therapy for a subject, the method involving identifying a subject as having central nervous system injury (e.g., a spinal cord injury) or other type of condition causing neuron injury (e.g., stroke or myocardial infarction), and administering to the subject a therapeutic composition of the invention.

### Methods for Evaluating Therapeutic Efficacy

In one approach, the efficacy of the treatment is evaluated by measuring, for example, the biological function of the treated organ (e.g., spinal cord function, nerve cell function, cardiac cell function, plasticity). Such methods are standard in the art and are described, for example, in the Textbook of Medical Physiology, Tenth edition, (Guyton et al., W.B. Saunders Co., 2000). In particular, a method of the present invention, increases the biological function of a tissue or organ by at least 5%, 10%, 20%, 40%, 50%, 60%, 70%, 80%, 90%, 100%, 150%, 200%, or even by as much as 300%, 400%, or 500%. Preferably, the tissue is nervous tissue and, preferably, the organ is the spinal cord.

Behavioral tests of recovery of function may also be used to evaluate treatment efficacy, including, for example, limb movement, joint movement, recovery of autonomic function, muscle strength, sensory gain and/or reduction of neuropathic pain.

In another approach, the therapeutic efficacy of the methods of the invention is assayed by measuring an increase in cell, neuron, or neurite number in the treated or transplanted tissue or organ as compared to a corresponding control tissue or organ (e.g., a tissue or organ that did not receive treatment). Preferably, the cell, neuron, or neurite number in a tissue or organ is increased by at least 5%, 10%, 20%, 40%, 60%, 80%, 100%, 150%, or 200% relative to a corresponding tissue or organ. Methods for assaying cell proliferation are known to the skilled artisan and are described, for example, in Bonifacino et al., (Current Protocols in Cell Biology Loose-leaf, John Wiley and Sons, Inc., San Francisco, Calif.). For example, assays for cell proliferation may involve the measurement of DNA synthesis during cell replication. In one embodiment, DNA synthesis is detected using labeled DNA precursors, such as [³H]-Thymidine or 5-bromo-2*-deoxyuridine [BrdU], which are added to cells (or animals) and then the incorporation of these precursors into genomic DNA during the S phase of the cell cycle (replication) is detected (Ruefli-Brasse et al., Science 302(5650):1581-4, 2003; Gu et al., Science 302 (5644):445-9, 2003).

In another approach, efficacy is measured by detecting an increase in the number of viable cells, neurons, or neurites present in a tissue or organ relative to the number present in an untreated control tissue or organ, or the number present prior to treatment. Assays for measuring cell viability are known in the art, and are described, for example, by Crouch et al. (J. Immunol. Meth. 160, 81-8); Kangas et al. (Med. Biol. 62, 338-43, 1984); Lundin et al., (Meth. Enzymo1.133, 27-42, 1986); Petty et al. (Comparison of J. Biolum. Chemilum. 10, 29-34, 0.1995); and Cree et al. (AntiCancer Drugs 6: 398-404, 1995). Cell viability can be assayed using a variety of methods, including MTT (3-(4,5-dimethylthiazolyl)-2,5-diphenyltetrazolium bromide) (Barltrop, Bioorg. & Med. Chem. Lett. 1: 611, 1991; Cory et al., Cancer Comm 3, 207-12, 1991; Paull J. Heterocyclic Chem. 25, 911, 1988). Assays for cell viability are also available commercially. These assays include but are not limited to CELLTITER-GLO® Luminescent Cell Viability Assay (Promega), which uses luciferase technology to detect ATP and quantify the health or number of cells in culture, and the CellTiter-Glo® Luminescent Cell Viability Assay, which is a lactate dehyrodgenase (LDH) cytotoxicity assay (Promega).

In some cases, therapeutic efficacy of spinal cord injury is assessed by measuring improvement in neural function. See, e.g., International Standards for the Neurological Classification of Spinal Cord Injury (ISNCSCI), J Spinal Cord Med. 2011 November; 34(6):547-54, incorporated herein by reference. As described in detail above, the ISNCSCI classification system is based on neurological responses, and is characterized by five categories on the ASIA Impairment Scale: “A-E,” which range from “complete” spinal cord injury, wherein no motor or sensory function is preserved in the sacral segments S4-S5 to “normal,” wherein motor and sensory scores are normal. Thus, in some cases, an improvement in neural function is determined by an elevation toward “E” (normal), as determined by the ISNCSCI classification system. For example, improvement in neural function is determined by an elevation of A to B, B to C, C to D, or D to E. Alternatively, improvement in neural function is assessed by complete or partial recovery of motor/sensory function.

In some cases, the methods described herein result in complete recovery of motor/sensory function. In other cases, the methods described herein result in partial recovery of motor/sensory function, e.g., when complete recovery of spinal cord injury is not possible. For example, the methods described herein result in 1%-100% recovery of motor/sensory function after spinal cord injury, e.g., about 2%, about 3%, about 4%, about 5%, about 6%, about 7%, about 8%, about 9%, about 10%, about 15%, about 20%, about 25%, about 30%, about 35%, about 40%, about 45%, about 50%, about 60%, about 70%, about 80%, about 90%, about 95% or about 99% recovery of motor/sensory function after spinal cord injury.

Alternatively, or in addition, therapeutic efficacy is assessed by measuring a reduction in apoptosis. Apoptotic cells are characterized by characteristic morphological changes, including chromatin condensation, cell shrinkage and membrane blebbing, which can be clearly observed using light microscopy. The biochemical features of apoptosis include DNA fragmentation, protein cleavage at specific locations, increased mitochondrial membrane permeability, and the appearance of phosphatidylserine on the cell membrane surface. Assays for apoptosis are known in the art. Exemplary assays include TUNEL (Terminal deoxynucleotidyl Transferase Biotin-dUTP Nick End Labeling) assays, caspase activity (specifically caspase-3) assays, and assays for fas-ligand and annexin V. Commercially available products for detecting apoptosis include, for example, Apo-ONE® Homogeneous Caspase-3/7 Assay, FragEL TUNEL kit (ONCOGENE RESEARCH PRODUCTS, San Diego, Calif.), the ApoBrdU DNA Fragmentation Assay (BIOVISION, Mountain View, Calif.), and the Quick Apoptotic DNA Ladder Detection Kit (BIOVISION, Mountain View, Calif.).

### Kits or Pharmaceutical Systems

The present compositions may be assembled into kits or pharmaceutical systems for use in neuron or neurite growth and/or migration. The compositions of the kits or pharmaceutical systems may be used for treating a Central Nervous System (CNS) injury (e.g., spinal cord injury) or other trauma (e.g., stroke, myocardial infarction). Kits or pharmaceutical systems according to this aspect of the invention comprise a carrier means, such as a box, carton, tube or the like, having in close confinement therein one or more container means, such as vials, tubes, ampoules, bottles and the like. The kits or pharmaceutical systems of the invention may also comprise associated instructions for using the agents of the invention. Kits of the invention include at least one or more agents with 4-sulfatase activity (e.g., human arylsulfatase B; bacterial 4-sulfatase). The kit may include instructions for administering the alkylating agent in combination with one or more agents with 4-sulfatase activity. Methods for measuring the efficacy of agents with 4-sulfatase activity are known in the art and are described herein.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### 4-Sulfatase is Effective for Increasing Neuronal Growth and/or Migration

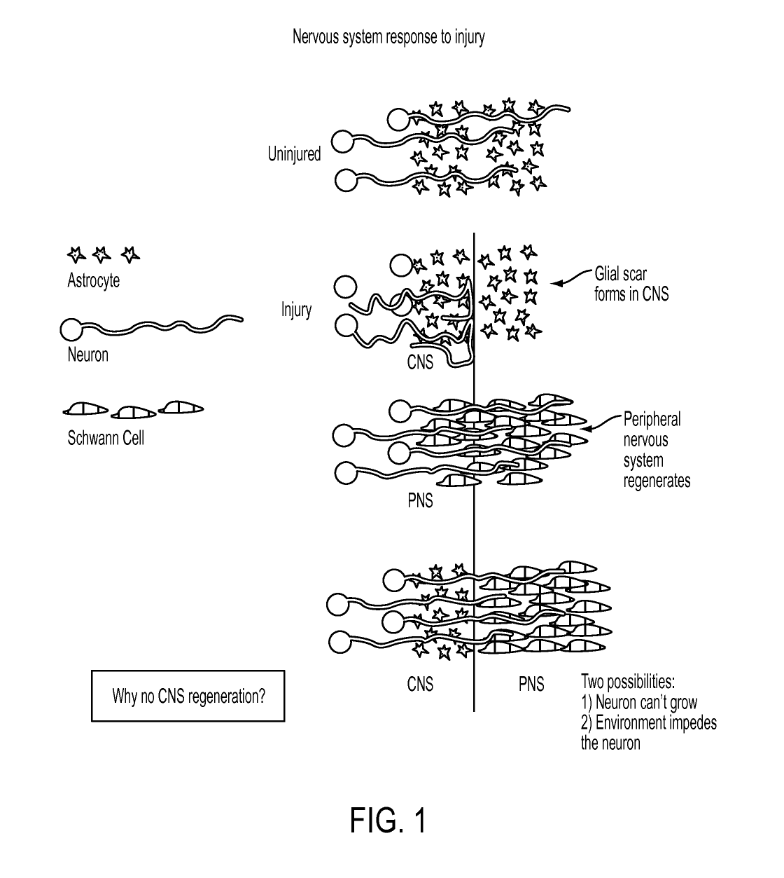

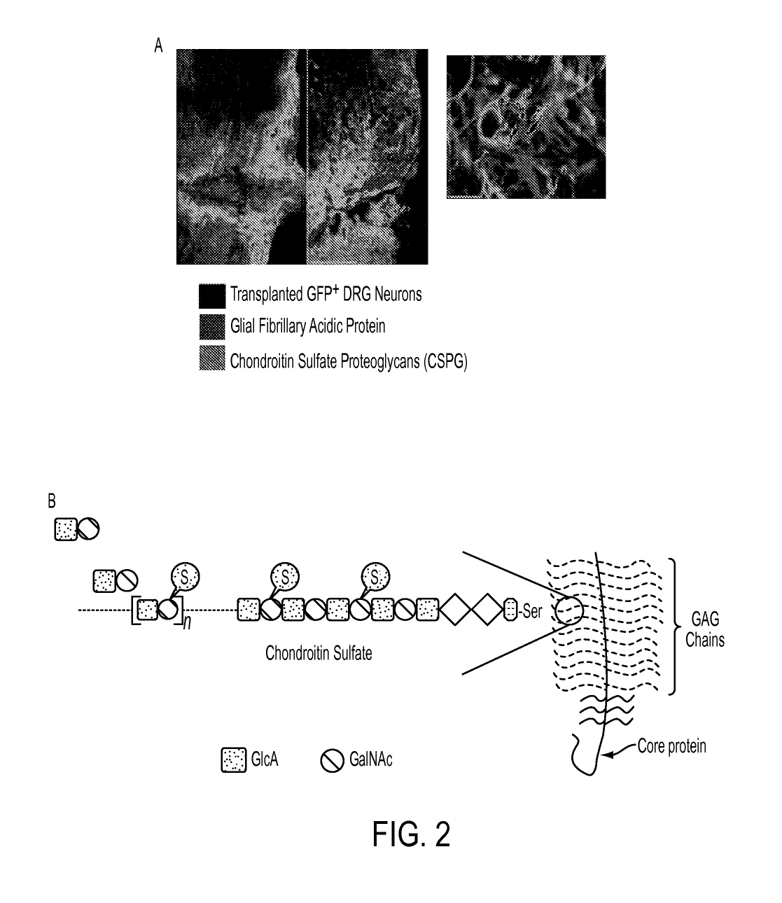

In contrast to peripheral nervous system (PNS), central nervous system (CNS) does not regenerate following injury (FIG. 1). Without being bound to a particular theory, formation of glial scar at the site of trauma prevents neuronal growth and/or creates an environment that inhibits neuronal growth. Chondroitin sulfate proteoglycans (CSPG) are present at the site of CNS injury (FIG. 2). Chondroitin sulfate proteoglycans consist of a protein core and GAG side chains of alternating GlcA and GalNAc monomers, which may be modified by a sulfate moiety.

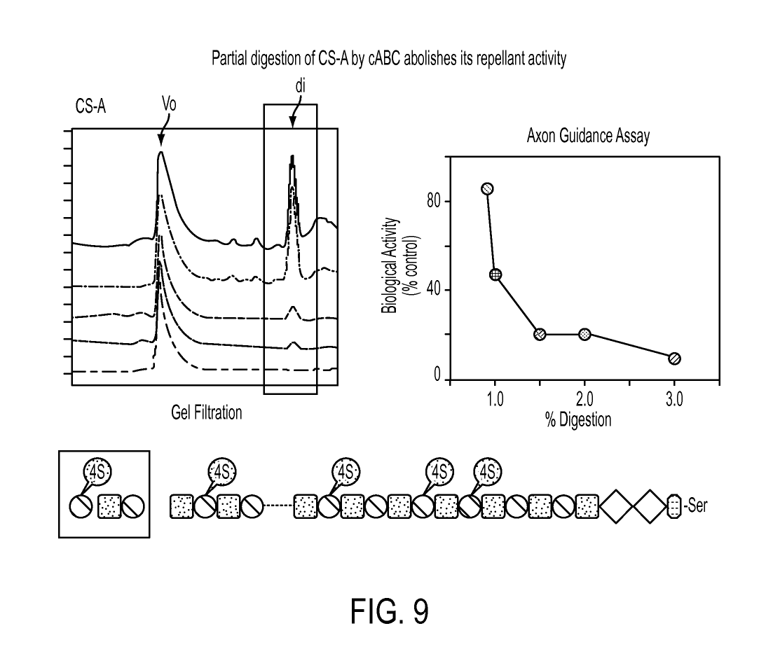

Inhibitory action of GAG chains resides in their sulfation, as inhibition of sulfation also promotes axonal regrowth in cell culture models. For example, partial digestion is effective for decreasing repellant activity of neurons by chondroitin sulfate (FIG. 9). Blocking steps of chondroitin sulfate biosynthesis to inhibit biosynthesis of chondroitin sulfate has been proposed to treat or prevent glial scar formation and/or increase neuronal growth at sites of injury (FIG. 6). However, published studies conflict regarding the effect of chondroitin sulfate modifications on neuronal growth (FIG. 7).

As shown herein, it has been discovered that the 4-sulfate unit on N-acetylgalactosamine is important for axonal regrowth. The results demonstrate that reducing the amount of 4-sulfation in culture promoted axonal regrowth, for example, compared to 6-sulfation where little or no effect on axonal regrowth was observed.

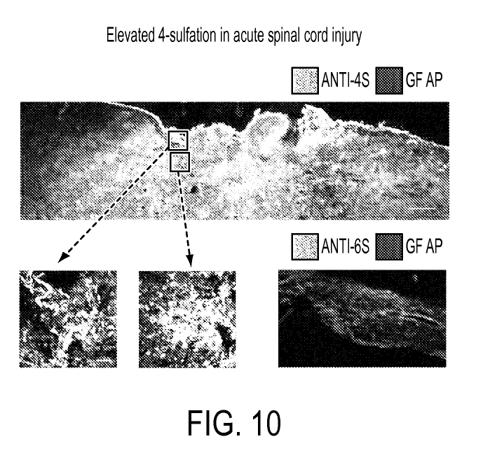

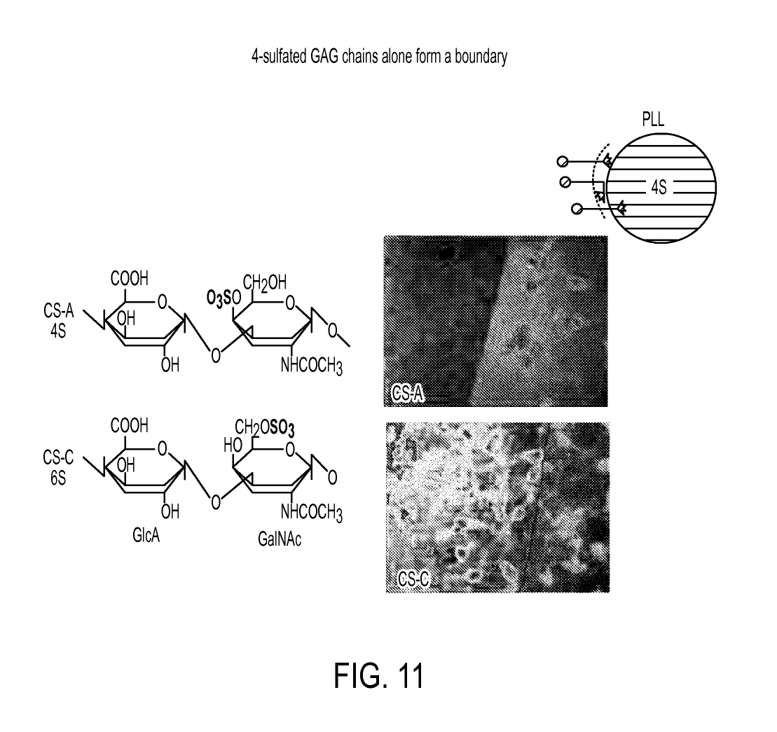

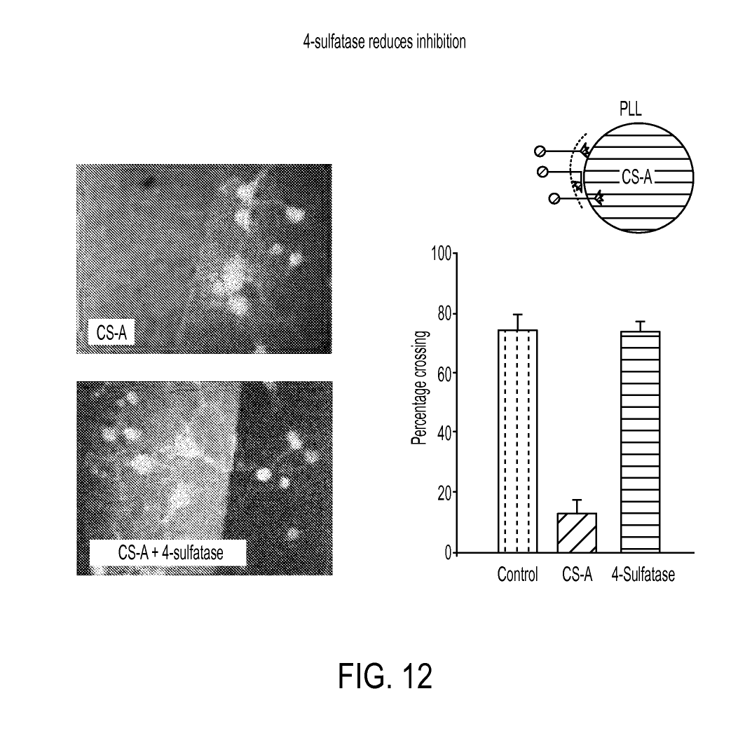

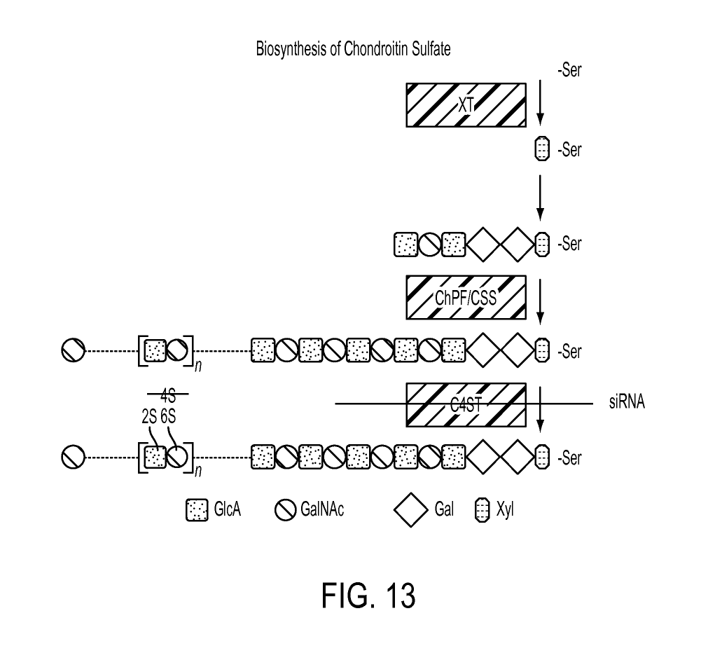

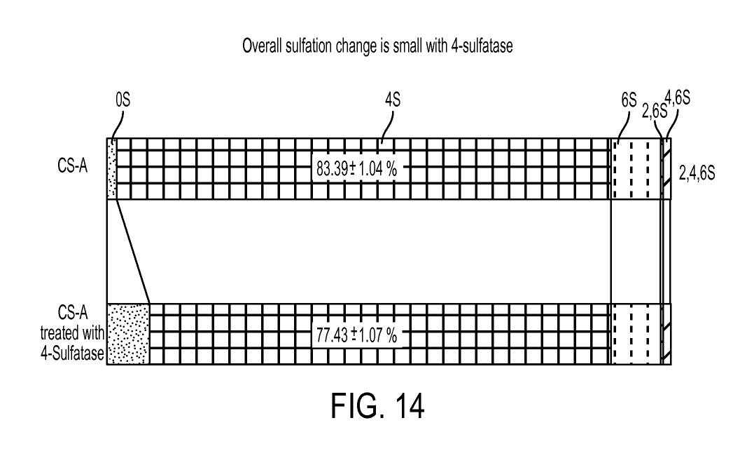

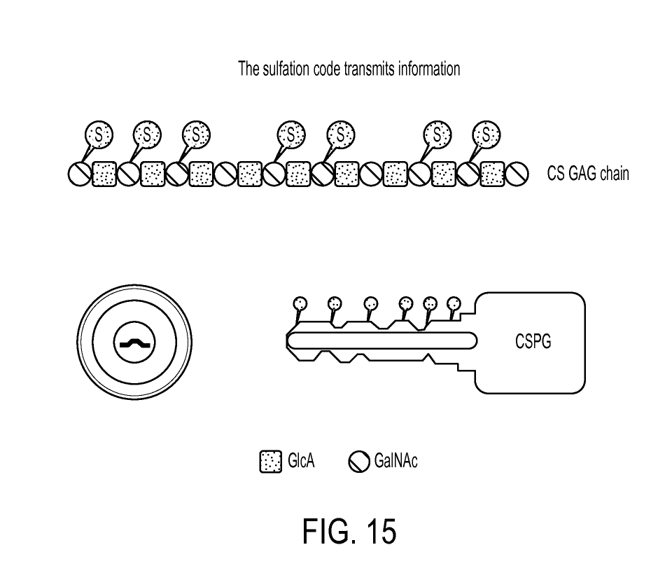

Increased staining of 4-sulfation of chondroitin sulfate was detected at the site of injury (FIG. 10). Additionally, it was observed that 4-S chondroitin sulfate chains formed a boundary for neurons and/or has an activity that repelled neurons (FIG. 11). Treatment of chondroitin sulfate chains with bacterial 4-sulfatase decreased the neuron repellant and/or boundary activity of chondroitin sulfate (FIG. 12). Thus, blocking 4-S modification of chondroitin sulfate has the potential to treat or prevent glial scar formation and/or increase neuronal growth at sites of injury (FIG. 13). When chondroitin sulfate was treated with 4-sulfatase, the change in overall sulfation was small (FIG. 14). Treatment of chondroitin sulfate with 4-sulfatase decreased overall sulfation by about 6% compared to a control without treatment. Without being bound to a particular theory, sulfation of chondroitin may provide signals affecting neuronal growth and, the 4-S modification of chondroitin sulfate may provide a signal or activity that prevents or decreases neuronal growth (FIG. 15). In particular, a prominent role for the 4-sulfation of the N-acetlygalactosamine sugar at the so-called non-reducing terminal of the sugar chain (the terminal that is furthest from the protein core) was shown.

A human 4-sulfatase called arylsulfatase B (ARSB) removes only the 4-sulfate group from the non-reducing ends of chondroitin sulfate glycosaminoglycan chains and can act on long chain glycosaminoglycan substrates. This is in contrast to the activity of bacterial sulfatases which do not remove the 4-sulfate group from long GAG chains effectively, and which acts only on short chain glycosaminoglycan chain substrates (e.g., disaccharides). While bacterial 4-sulfatase can remove 4-sulfate group from GlcA β1-3GalNAc (4S), ARSB is not able to do so because of the presence of GlcA at the non-reducing end. Thus, GalNAc(4S) should be located at the non-reducing end of the GAG chain to be a substrate for ARSB.

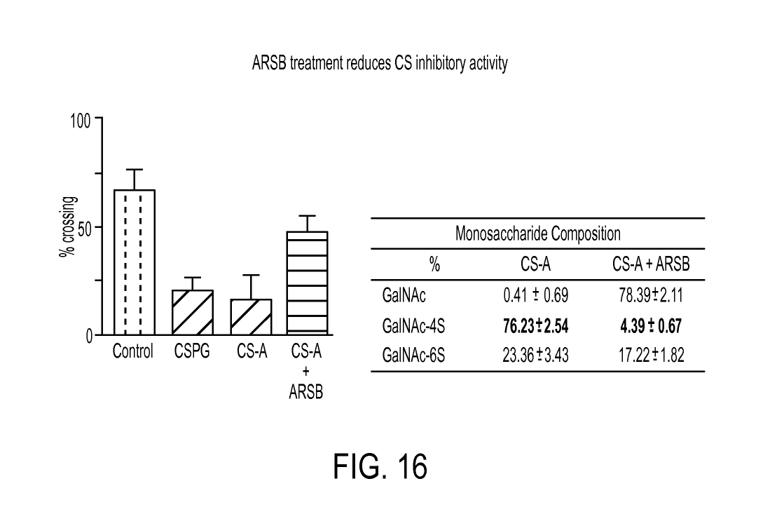

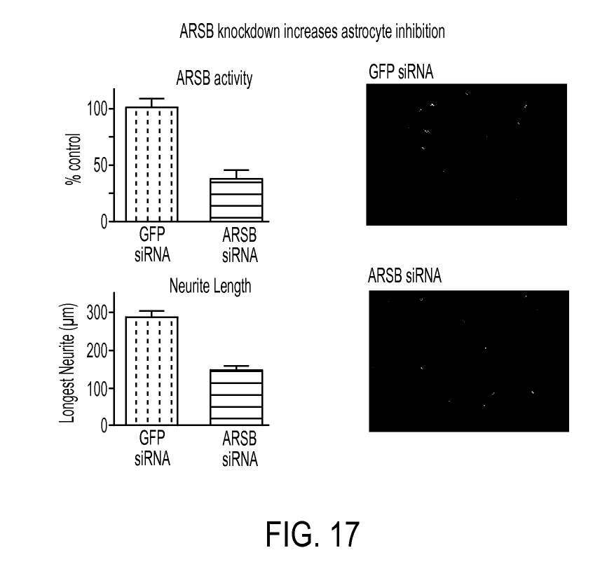

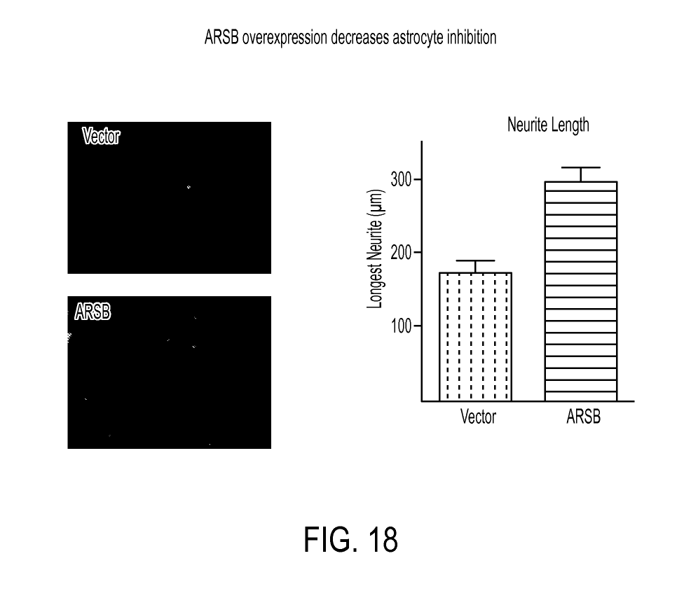

Application of ARSB in culture improved neuronal regrowth in response to CSPGs compared to control without treatment (FIG. 16). Interestingly, knockdown of arylsulfatase B (ARSB) expression decreased neuronal growth and/or increased astrocyte inhibition compared to control as measured by neurite length (FIG. 17). Whereas, overexpression of arylsulfatase B (ARSB) expression increased neuronal growth and/or decreased astrocyte inhibition compared to control as measured by neurite length (FIG. 18).

### Example 2

### ARSB Promoted Axonal Regeneration In Vivo

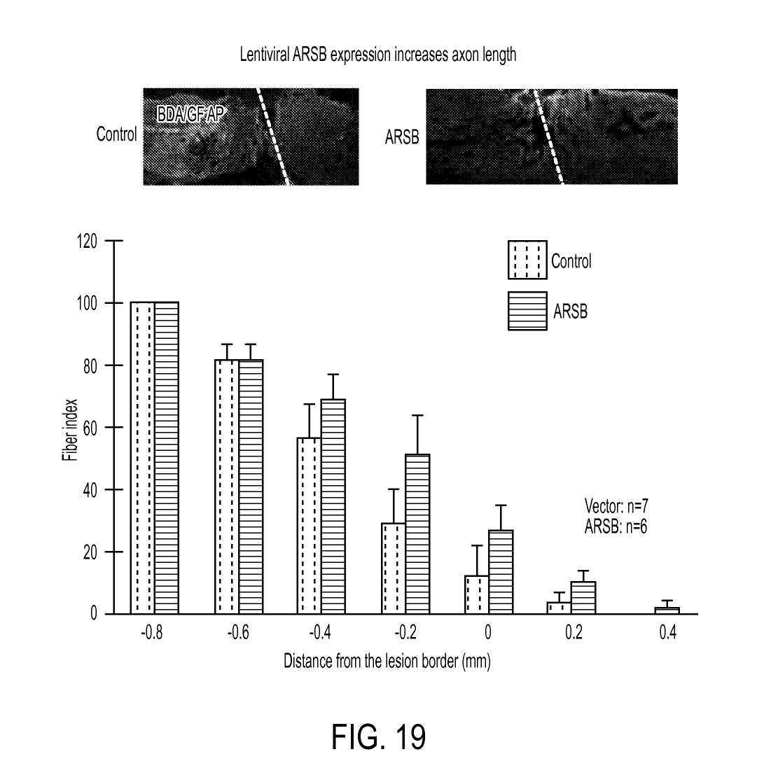

A lentiviral vector that expressed ARSB was created for in vivo experiments in mice. It was found that injection of lentivirus with the ARSB expression vector into the lesion of mouse spinal cord promoted axonal regeneration. Lentiviral arylsulfatase B (ARSB) expression increased axon length at and around the lesion border compared to control as measured by fiber index (FIG. 19).

These results indicate that preventing, inhibiting, or removing the 4-sulfate group from chondroitin sulfate (e.g., increasing its degradation and/or decreasing chondroitin 4-sulfate biosynthesis) is effective for treating nervous system injury, treating glial scarring, and/or increasing neuron or neurite growth and migration. Additionally, these results indicate that 4-sulfatases, such as arylsulfatase B, are effective for treating nervous system injury, treating glial scarring, and/or increasing neuron or neurite growth and migration.

### Example 3

### ARSB Improves Locomotor Function in Mice

The following example demonstrates that arysulfatase B (ARSB) enhances recovery after spinal cord injury in mice as measured by improvements in locomotor recovery assessed by the Basso Mouse Scale (BMS) (Apostolova I, et al. 2006 J Neurosci, 26:7849-7859; Basso D M, et al. 2006 J Neurotrauma 23: 635-659, each of which are incorporated herein by reference).

### Spinal Injury and Intraspinal Injection

After anaesthetizing the mice with an intraperitoneal injection of Ketamine (160 mg/kg (Butler Schein Animal Health, Chicago, Ill.) and Xylazine (24 mg/kg, Butler Schein Animal Health, Chicago, Ill.), 0.1 ml of 0.125% Bupivacaine (Hospira, Inc., Lake Forest, Ill.) is injected around the incision site to provide local anesthesia. A three cm skin incision along the median line on the back of the animals is made. Laminectomy is performed with Mouse Laminectomy Forceps (Fine Science Tools, Heidelberg, Germany) at the T7-T9 level, followed by a mechanically controlled compression injury using a mouse spinal cord compression device (Yoo M et al., 2013 PLOS ONE, 8(3):e57415, incorporated herein by reference). The spinal cord is compressed for 0.5 seconds for the moderate compression injury and 1 second for the severe compression injury by a timed current through the electromagnetic drive.

Human ARSB is expressed and purified to homogeneity as described (Dierks T, et al., 1998 FEBS Lett, 423: 61-65, incorporated herein by reference) Immediately after spinal cord injury, one ml of ARSB (10 U/ml) in Tris-buffered saline, PH 7.3 is injected at the injury site and 0.5 mm rostral and caudal to this site. Specifically, a 33-gauge needle connected to a 5 ml Hamilton syringe (Hamilton, Reno, Nev.) in a stereotactic micromanipulator (Narishige, N.Y., N.Y.) is utilized. ARSB (10 units/ml; 10 mice) or the control TBS solution (10 mice) is injected 1 mm deep into the cord midline (1 ml at the lesion center and 1 ml each at sites 0.5 mm rostral and caudal to the lesion center), with each injection lasting for 7 min. After injection, the skin is closed with wound clips.

### Locomotor Assessment

Locomotor function is assessed by the Basso Mouse Scale (BMS) score one week before and each week after injury. The mice are assessed for locomotor activity for 12 weeks. For assessment of the BMS, the mice are allowed to move in an open field, 1 meter in diameter, for 5 min The hindlimb movements are observed and scored according to the BMS scale. Statistical analyses are performed.

Mice injected with ARSB exhibit an improvement in locomotor function, as measured by the BMS compared to the Tris-buffered saline control group. For example, ARSB improves breathing function, diaphragm function, arm sensory/functional aspects, shoulder sensory/functional aspects, wrist sensory/functional aspects, hand sensory/functional aspects, finger sensory/functional aspects, abdominal muscle sensory/functional aspects, leg sensory/functional aspects, hip sensory/functional aspects, feet sensory/functional aspects, toe sensory/functional aspects, bladder function, anal function, or sexual function.

### Example 4

### ARSB Increases Axon Length in a Controlled Cortical Impact Injury Model

Controlled cortical impact injury is performed as described in Yi et al., 2012 The Journal of Comparative Neurology, 520:3295-3313, incorporated herein by reference. Specifically, mice are anesthetized with isoflurane (4% for induction, 2-3% for maintenance), and their heads are securely placed in a mouse stereotaxic frame (Digital Lab Standard, Stoelting, Wood Dale, Ill.). Body temperature is kept constant by using an isothermal heating pad (Stoelting) throughout surgery. Respiration rate is visually monitored during the period when mice are under anesthesia. Following cleansing with alcohol and iodine, an incision is made over the forehead, and the scalp is reflected to expose the skull. A 4 mm diameter craniotomy is made over the left hemisphere with a surgical drill (Stoelting), and the bone flap is carefully removed with fine forceps. Mice are injured over the left somatosensory cortex (0 bregma, 2 mm lateral to the suture line) at an impact depth of 1 mm with a 2 mm diameter round impact tip (speed 3.6 msec, dwell time 100 msec) by using an electromagnetically driven controlled cortical impact injury device (Impact One stereotaxic impactor CCI, Leica Microsystems, Wetzlar, Germany; Brody et al., 2007). The angle of the impactor tip is set so the bottom is aligned flat with the dura, typically 10-15°. The dura remains intact following craniotomy Impact causes tearing of the dura with occasional subdural hemorrhages and mild edema. Following injury, the bone flap is replaced, and the scalp is sutured closed. An antiseptic ointment (Neosporin) is applied over the sutured area. Mice are given a saline bolus (i.p., 100 μl) and transferred to a thermal heating pad until fully recovered from anesthesia. Mice are under isoflurane for no longer than 15 minutes. Sham-injured animals receive the same surgery without the impact injury.

A vector that has been engineered to expresses ARSB is administered to the spinal cord lesion, and the lesion is evaluated daily for a period of 12 weeks for an increase in the number and length of axons in proximity to the spinal cord lesion. An increase in the number and/or length of axons at or near the spinal cord lesion indicates that ARSB successfully treated the spinal cord injury.

### Example 5

### ARSB Improves Locomotor Function in Humans

Human ARSB is expressed and purified to homogeneity as described (Dierks T, et al., 1998 FEBS Lett, 423: 61-65, incorporated herein by reference). After a human receives a spinal cord injury, 1 mg of ARSB (10 U/ml) in Tris-buffered saline, PH 7.3 is injected at the injury site and 0.5 mm rostral and caudal to this site. Of course, the therapeutically effective amount may be optimized via upward or downward adjustments, as is routinely done in such treatment protocols, depending upon the specific condition and severity thereof.

Human locomotor function is assessed according to International Standards for the Neurological Classification of Spinal Cord Injury (ISNCSCI; J Spinal Cord Med. 2011 November; 34(6):547-54, incorporated herein by reference) immediately before and every week after injection. ARSB injection improves the function of various tissues and/or organs. For example, ARSB injection results in an improvement in cognitive skills, breathing function, diaphragm function, arm function, shoulder function, wrist function, hand function, finger function, abdominal muscle function, leg function, hip function, feet function, toe function, bladder function, anal function, and/or sexual function. ARSB injection also results alleviate pain and/or paralysis.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

### REFERENCES

The following documents are cited herein.
Bradbury et al., “Chondroitinase ABC promotes functional recovery after spinal cord injury.” Nature. 2002 Apr. 11; 416(6881):636-40.
Brown et al., “A sulfated carbohydrate epitope inhibits axon regeneration after injury.” Proc Natl Acad Sci USA. 2012 Mar. 27; 109(13):4768-73.
Clement et al., “The DSD-1 carbohydrate epitope depends on sulfation, correlates with chondroitin sulfate D motifs, and is sufficient to promote neurite outgrowth.” J Biol Chem. 1998; 273(43):28444-53.
Curinga et al., “Mammalian-produced chondroitinase AC mitigates axon inhibition by chondroitin sulfate proteoglycans.” J Neurochem. 2007; 102(1):275-88.
Gama et al., “Sulfation patterns of glycosaminoglycans encode molecular recognition and activity.” Nat Chem Biol. 2006; 2(9):467-73.
Gilbert et al. “CS-4,6 is differentially upregulated in glial scar and is a potent inhibitor of neurite extension.” Mol Cell Neurosci. 2005; 29(4):545-58.
Iseda et al. “Single, high-dose intraspinal injection of chondroitinase reduces glycosaminoglycans in injured spinal cord and promotes corticospinal axonal regrowth after hemisection but not contusion.” J Neurotrauma. 2008; 25(4):334-49.
Wang et al. “Chondroitin-4-sulfation negatively regulates axonal guidance and growth.” J Cell Sci. 2008; 121(Pt 18):3083-91.

1. A method of treating or preventing a central nervous system injury in a subject, the method comprising administering to said subject a therapeutically effective amount of an agent that reduces the amount or activity of a 4-sulfated GalNAc in a chondroitin glycosaminoglycan chain, thereby treating the central nervous system injury.

2. A method of treating or preventing a glial scar in a subject, the method comprising administering to said subject a therapeutically effective amount of an agent that reduces the amount or activity of a 4-sulfated GalNAc in a chondroitin glycosaminoglycan chain, thereby treating the glial scar.

3. The method of claim 2, wherein the subject has had or is at risk of having a central nervous system injury, stroke, or myocardial infarction.

4. The method of claim 1, wherein the agent increases neuron or neurite growth, proliferation, or migration.

5. A method of increasing neuron or neurite growth, proliferation, or migration, the method comprising contacting a neuron with an effective amount of an agent that reduces the amount or activity of a 4-sulfated GalNAc in a chondroitin glycosaminoglycan chain, thereby increasing neuron or neurite growth, proliferation, or migration.

6. The method of claim 5, wherein the neurite is a dendrite or axon.

7. The method of claim 5, wherein the neuron or neurite growth, proliferation, or migration is in a subject.

8. The method of claim 7, wherein the subject has had or is at risk of having a central nervous system injury, stroke, or myocardial infarction.

9. The method of claim 1, wherein the central nervous system injury is a spinal cord injury, contusion injury to the central nervous system, or non-contusion injury to the central nervous system.

10.-13. (canceled)

14. The method of claim 1, wherein the 4-sulfated GalNAc is at a non-reducing end of a chondroitin glycosaminoglycan chain.

15. The method claim 1, wherein the 4-sulfated GalNAc reduces neuron or neurite growth, proliferation, or migration.

16. The method of claim 1, wherein the agent has 4-sulfatase activity.

17. The method of claim 16, wherein the agent having 4-sulfatase activity is a human or bacterial GalNAc 4-sulfatase.

18. The method of claim 17, wherein the human GalNAc 4-sulfatase is human arylsufatase B.

19. The method of claim 18, wherein said human arylsufatase B does not remove the 4-sulfate group from GlcA β1-3GalNAc (4S).

20. The method of claim 1, wherein the agent reduces 4-sulfation of a chondroitin glycosaminoglycan chain.

21. The method of claim 1, wherein said method improves breathing function, diaphragm function, arm function, shoulder function, wrist function, hand function, finger function, abdominal muscle function, leg function, hip function, feet function, toe function, bladder function, anal function, or sexual function.

22. The method of claim 1, wherein said method increases the number of axons or axonal length in said subject.

23. The method of claim 1, wherein the agent inhibits a chondroitin sulfate transferase.

24. The method of claim 23, wherein the chondroitin sulfate transferase is selected from the group consisting of chondroitin 4-O-sulfotransferase 1/CHST11, chondroitin 4-O-sulfotransferase 2/CHST12, chondroitin 4-O-sulfotransferase 3/CHST13, dermatan 4-O-sulfotransferase 1/CHST14, chondroitin 6-O-sulfotransferase 1/CHST3, uronyl 2-sulfotransferase/UST, carbohydrate (N-acetylgalactosamine 4-sulfate 6-O) sulfotransferase 15/CHST15.

25. The method of claim 22, wherein the agent is an inhibitory nucleic acid molecule.

26. The method of claim 23, wherein the inhibitory nucleic acid molecule is selected from the group consisting of an antisense molecule, an siRNA, and an shRNA.

27. The method of claim 22, wherein the agent specifically binds a 4-sulfated GalNAc.

28. The method of claim 22, wherein the agent is an antibody or fragment thereof.

29. The method of claim 28, wherein the antibody is monoclonal or polyclonal.
